(19) United States
(12) Patent Application Publication
(10) Pub. No.: US 2015/0133744 A1
(43) Pub. Date: May 14, 2015

(54) LUTEUM FUNCTION EVALUATION APPARATUS, LUTEUM FUNCTION EVALUATION SYSTEM, AND CONTROL METHOD THEREOF

(71) Applicant: OMRON Healthcare Co., Ltd., Muko-shi, Kyoto (JP)

(72) Inventors: Tatsuya Kobayashi, Kyoto (JP); Shigemi Kamegawa, Kyoto (JP)

(21) Appl. No.: 14/398,193

(22) PCT Filed: Apr. 25, 2013
(86) PCT No.: PCT/JP2013/062207

§ 371 Date: Oct. 31, 2014

(30) Foreign Application Priority Data
May 1, 2012 (JP) 2012-104812

(51) Int. Cl. A61B 10/00; A61B 5/00; A61B 5/01

(52) U.S. Cl. CPC A61B 10/0012; A61B 5/01; A61B 5/7278; A61B 5/7282; A61B 5/0008; A61B 5/7475; A61B 5/742; A61B 5/0022; A61B 5/6898; A61B 2010/0019; A61B 2010/0029
USPC 600/301

(57) ABSTRACT

A luteum function evaluation apparatus includes a body temperature obtainment unit that obtains basal body temperature data including information of basal body temperature measurement values, a menstrual cycle obtainment unit that obtains information of a menstruation start day and a menstruation end day corresponding to the menstruation start day, an evaluation unit that evaluates luteum function based on the basal body temperature measurement values, and an output unit that outputs a luteum function evaluation result from the evaluation unit.

### BACKGROUND OF THE INVENTION

1. Field of the Invention

The present invention relates to apparatuses that evaluate luteum function, and particularly relates to apparatuses that evaluate luteum function based on a basal body temperature.

The present invention also relates to programs for causing a computer to execute a method that evaluates luteum function based on a basal body temperature.

2. Description of the Related Art

Services for predicting a woman's ovulation days, menstruation days, and so on have thus far been offered as services related to the woman's basal body temperature. JP 2008-264352A, for example, discloses this type of technique, in which a biphasic nature of the body temperature during a menstrual cycle is estimated based on body temperature detection data obtained over multiple days.

Meanwhile, JP 2010-502338A discloses providing information regarding the fertility of a female mammal by taking a plurality of temperatures from the female mammal, obtaining a representative temperature value that is neither a maximum value nor a minimum value from the plurality of temperatures, and analyzing that representative temperature value.

However, the aforementioned conventional techniques do not provide information related to the evaluation of luteum function. Accordingly, it has been necessary for a user to request a doctor's examination in the case where the user desires to obtain knowledge regarding her luteum function. For this reason, there has been demand for a technique capable of evaluating luteum function with ease.

### SUMMARY OF THE INVENTION

Accordingly, preferred embodiments of the present invention provide an apparatus that enables a user to easily obtain knowledge regarding luteum function.

A luteum function evaluation apparatus according to an aspect of various preferred embodiments of the present invention includes a body temperature obtainment unit that obtains basal body temperature data of a measurement subject including information of basal body temperature measurement values from a plurality of days, a menstrual cycle obtainment unit that obtains information of a menstruation start day included in the plurality of days and a menstruation end day corresponding to the menstruation start day, an evaluation unit that evaluates luteum function based on a plurality of basal body temperature measurement values measured on different days within a period spanning from the menstruation start day to the menstruation end day, and an output unit that outputs a luteum function evaluation result from the evaluation unit.

The luteum function evaluation apparatus according to this aspect of various preferred embodiments of the present invention evaluates luteum function based on the basal body temperature measurement values of the measurement subject from a plurality of days and outputs the evaluation result. Through this, the luteum function evaluation result is presented to the measurement subject. Accordingly, a user can easily obtain knowledge regarding her luteum function.

In the luteum function evaluation apparatus according to an aspect of various preferred embodiments of the present invention, the evaluation unit preferably evaluates the luteum function according to a plurality of evaluation levels including at least “normal” and “insufficient”.

The luteum function evaluation apparatus according to this aspect of various preferred embodiments of the present invention can evaluate the measurement subject's luteum function as normal or as not normal (insufficient). Accordingly, a user can easily obtain knowledge as to whether or not her luteum function is normal.

Note that in the present specification, luteum function being “insufficient” includes all states in which luteum function is not evaluated as normal, regardless of whether or not ovulation is occurring, for example.

In the luteum function evaluation apparatus according to an aspect of various preferred embodiments of the present invention, in the case where the luteum function has been evaluated as “insufficient”, the evaluation unit preferably further estimates whether or not ovulation is occurring based on the plurality of basal body temperature measurement values, and preferably further divides the evaluation of insufficient into a plurality of classes including at least “with ovulation” and “no ovulation”.

The luteum function evaluation apparatus according to this aspect of various preferred embodiments of the present invention preferably estimates whether or not ovulation is occurring in the case where the measurement subject's luteum function has been evaluated as insufficient, and then indicates the result of the estimation in the luteum function evaluation. Accordingly, the user can easily obtain knowledge as to whether or not ovulation has occurred.

In the luteum function evaluation apparatus according to an aspect of various preferred embodiments of the present invention, the evaluation unit preferably derives a current menstrual cycle that starts on the menstruation start day based on the information of the menstruation start day and the menstruation end day, preferably derives a phasic nature indicating whether or not a basal body temperature curve includes a high temperature phase and a low temperature phase and is thus biphasic based on the plurality of basal body temperature measurement values, and preferably evaluates the luteum function based on a number of days in the current menstrual cycle and the phasic nature of the basal body temperature curve.

In the luteum function evaluation apparatus according to an aspect of various preferred embodiments of the present invention, the evaluation unit preferably derives a number of days in the high temperature phase of the current menstrual cycle based on the plurality of basal body temperature measurement values, and preferably evaluates the luteum function based on the number of days in the current menstrual cycle, the phasic nature of the basal body temperature curve, and the number of days in the high temperature phase.

In the luteum function evaluation apparatus according to an aspect of various preferred embodiments of the present invention, the evaluation unit preferably derives a high temperature phase rise end day and a high temperature phase fall start day based on the plurality of basal body temperature measurement values, and preferably evaluates the luteum function based on the number of days in the current menstrual cycle, the phasic nature of the basal body temperature curve, the number of days in the high temperature phase, the high temperature phase rise end day, and the high temperature phase fall start day.

In the luteum function evaluation apparatus according to an aspect of various preferred embodiments of the present invention, based on the plurality of basal body temperature measurement values, the evaluation unit preferably derives a high-temperature sufficient consecutive number of days that is a number of consecutive days in the low temperature phase in which the basal body temperature measurement value is higher than a predetermined temperature, a low temperature phase instability level that is an index indicating a level of fluctuation in the basal body temperature measurement value during the low temperature phase, and a high temperature phase stability level that is an index indicating a stability level of the basal body temperature measurement value during the high temperature phase, and preferably evaluates the luteum function based on the number of days in the current menstrual cycle, the phasic nature of the basal body temperature curve, the number of days in the high temperature phase, the high temperature phase rise end day, the high temperature phase fall start day, the high-temperature sufficient consecutive number of days, the low temperature phase instability level, and the high temperature phase stability level.

In the luteum function evaluation apparatus according to an aspect of various preferred embodiments of the present invention, based on the plurality of basal body temperature measurement values, the evaluation unit preferably derives a low temperature phase standard upper limit value that is the highest temperature of the basal body temperature measurement values in a period spanning from a day after a predetermined first number of days following the menstruation start day to a final day of the low temperature phase, a low temperature phase standard lower limit value that is the lowest temperature of the basal body temperature measurement values in a period spanning from a day after the first number of days following the menstruation start day to the final day of the low temperature phase, a low temperature phase standard value that is an average value of the low temperature phase standard upper limit value and the low temperature phase standard lower limit value, a high temperature phase standard value that is an average value of the basal body temperature measurement values in the high temperature phase, and a post-rise maximum fall day temperature that is the basal body temperature measurement value on a day in the high temperature phase in which the basal body temperature measurement value has dropped the most from the basal body temperature measurement value on the previous day, and preferably evaluates the luteum function based on the number of days in the current menstrual cycle, the phasic nature of the basal body temperature curve, the number of days in the high temperature phase, the high temperature phase rise end day, the high temperature phase fall start day, the high-temperature sufficient consecutive number of days, the low temperature phase instability level, the high temperature phase stability level, the low temperature phase standard upper limit value, the low temperature phase standard lower limit value, the low temperature phase standard value, the high temperature phase standard value, and the post-rise maximum fall day temperature.

In the luteum function evaluation apparatus according to an aspect of various preferred embodiments of the present invention, the evaluation unit preferably derives a current menstrual cycle starting on the menstruation start day based on the information of the menstruation start day and the menstruation end day, and preferably divides the current menstrual period into a plurality of classes including at least “epimenorrhagia”, “secondary amenorrhea”, and “oligomenorrhea”, for example, based on a number of days in the cycle.

The luteum function evaluation apparatus according to this aspect of various preferred embodiments of the present invention preferably determines an evaluation regarding a menstrual period in addition to the luteum function evaluation. Accordingly, the user can easily obtain knowledge as to whether or not her period indicates epimenorrhagia, secondary amenorrhea, or oligomenorrhea, for example.

In the luteum function evaluation apparatus according to an aspect of various preferred embodiments of the present invention, the menstrual cycle obtainment unit preferably further obtains a number of days in a previous menstrual cycle whose final day is the day before the menstruation start day, and the evaluation unit preferably evaluates whether or not the menstrual cycle is an abnormal menstrual cycle by comparing the number of days in the previous menstrual cycle with the current menstrual cycle.

The luteum function evaluation apparatus according to this aspect of various preferred embodiments of the present invention preferably determines an evaluation regarding a menstrual cycle in addition to the luteum function evaluation. Accordingly, the user can easily obtain knowledge as to whether or not her menstrual cycle is an abnormal menstrual cycle.

In the luteum function evaluation apparatus according to an aspect of various preferred embodiments of the present invention, based on the plurality of basal body temperature measurement values, the evaluation unit preferably segments the current menstrual cycle into a plurality of periods including at least a low temperature phase period and a high temperature phase period, and preferably evaluates the length of a follicular phase based on a result of the segmenting and the current menstrual cycle.

The luteum function evaluation apparatus according to this aspect of various preferred embodiments of the present invention preferably determines an evaluation regarding the length of a follicular phase in addition to the luteum function evaluation. Accordingly, the user can easily obtain knowledge as to whether her follicular phase is long or short.

The luteum function evaluation apparatus according to an aspect of various preferred embodiments of the present invention further includes a communication unit that receives, as the basal body temperature measurement value, a body temperature detected by an external thermometer from the external thermometer, and outputs the temperature to the body temperature obtainment unit, and a notifying unit that receives the luteum function evaluation result outputted by the output unit and notifies the user of the result.

The luteum function evaluation apparatus according to this aspect of various preferred embodiments of the present invention preferably receives, as the basal body temperature measurement value, a body temperature detected by the external thermometer from the external thermometer, and evaluates luteum function. Accordingly, the user can more easily obtain knowledge regarding her luteum function.

The luteum function evaluation apparatus according to an aspect of various preferred embodiments of the present invention further includes a sensor unit that detects a body temperature, and a notifying unit that receives the luteum function evaluation result outputted by the output unit and notifies a user of the result. In this example preferred embodiment of the present invention, the body temperature obtainment unit preferably obtains the body temperature detected by the sensor unit as the basal body temperature measurement value.

The luteum function evaluation apparatus according to this aspect of various preferred embodiments of the present invention preferably detects the body temperature using its own sensor unit, obtains the body temperature as the basal body temperature measurement value, and evaluates luteum function. Accordingly, the user can even more easily obtain knowledge regarding her luteum function.

A luteum function evaluation system according to another aspect of various preferred embodiments of the present invention includes a thermometer including a sensor unit that detects a body temperature and a first communication unit configured to send the body temperature detected by the sensor unit to the exterior as a basal body temperature measurement value; a terminal device including a second communication unit that receives the basal body temperature measurement value outputted by the first communication unit, a first network communication unit configured to send the received basal body temperature measurement value to a network and receive a luteum function evaluation result from the network, and a notifying unit that notifies a user of the luteum function evaluation result; and a luteum function evaluation apparatus including a body temperature obtainment unit that obtains basal body temperature data of a measurement subject including information of basal body temperature measurement values from a plurality of days, a menstrual cycle obtainment unit that obtains information of a menstruation start day included in the plurality of days and a menstruation end day corresponding to the menstruation start day, an evaluation unit that evaluates luteum function based on a plurality of basal body temperature measurement values measured on different days within a period spanning from the menstruation start day to the menstruation end day, an output unit that outputs a luteum function evaluation result from the evaluation unit, and a second network communication unit that receives the basal body temperature measurement values over the network and sends the luteum function evaluation result outputted by the output unit to the network.

The luteum function evaluation system according to this aspect of various preferred embodiments of the present invention evaluates luteum function based on the basal body temperature measurement values of the measurement subject from a plurality of days and outputs the evaluation result. Through this, the luteum function evaluation result is presented to the measurement subject. Accordingly, the user can easily obtain knowledge regarding her luteum function.

In the luteum function evaluation system according to an aspect of various preferred embodiments of the present invention, the terminal device preferably includes an operating unit that accepts input from the user and sends information of the menstruation start day and the menstruation end day inputted by the user from the first network communication unit to the network, and the menstrual cycle obtainment unit of the luteum function evaluation apparatus preferably obtains the information of the menstruation start day and the menstruation end day, sent by the terminal device, from the network via the second network communication unit.

In the luteum function evaluation system according to this aspect of various preferred embodiments of the present invention, the user can input the information of the menstruation start day and the menstruation end day by operating the terminal device, and the information preferably is then be sent to the luteum function evaluation apparatus over the network. Accordingly, the user can easily input the information of the menstruation start day and the menstruation end day, and the luteum function evaluation apparatus then evaluates luteum function based on the inputted information, which improves the accuracy of the luteum function evaluation.

In the luteum function evaluation system according to an aspect of various preferred embodiments of the present invention, the terminal device preferably includes a rendering unit capable of generating an image and a display unit capable of displaying an image, and the rendering unit preferably generates an image of a basal body temperature curve schematic diagram based on the luteum function evaluation result and causes the schematic diagram to be displayed in the display unit.

In the luteum function evaluation system according to this aspect of various preferred embodiments of the present invention, the basal body temperature curve schematic diagram is displayed in the display unit of the terminal device. Accordingly, the user can intuitively understand a characteristic of the basal body temperature curve, and can easily carry out appropriate actions such as being examined by a doctor.

A luteum function evaluation method according to another aspect of various preferred embodiments of the present invention includes a body temperature obtainment step of obtaining basal body temperature data of a measurement subject including information indicating basal body temperature measurement values from a plurality of days, a menstrual cycle obtainment step of obtaining information of a menstruation start day included in the plurality of days and a menstruation end day corresponding to the menstruation start day, an evaluation step of evaluating luteum function based on a plurality of basal body temperature measurement values measured on different days within a period spanning from the menstruation start day to the menstruation end day, and an output step of outputting a luteum function evaluation result from the evaluation step.

The luteum function evaluation method according to this aspect of various preferred embodiments of the present invention evaluates luteum function based on the basal body temperature measurement values of the measurement subject from a plurality of days and outputs the evaluation result. Through this, the luteum function evaluation result is presented to the measurement subject. Accordingly, the user can easily obtain knowledge regarding her luteum function.

A non-transitory computer-readable medium including a luteum function evaluation program according to another aspect of various preferred embodiments of the present invention is a program that causes a computer to execute a body temperature obtainment step of obtaining basal body temperature data of a measurement subject including information indicating basal body temperature measurement values from a plurality of days, a menstrual cycle obtainment step of obtaining information of a menstruation start day included in the plurality of days and a menstruation end day corresponding to the menstruation start day, an evaluation step of evaluating luteum function based on a plurality of basal body temperature measurement values measured on different days within a period spanning from the menstruation start day to the menstruation end day, and an output step of outputting a luteum function evaluation result from the evaluation step.

The non-transitory computer-readable medium including the luteum function evaluation program according to this aspect of various preferred embodiments of the present invention evaluates luteum function based on the basal body temperature measurement values of the measurement subject from a plurality of days and outputs the evaluation result. Through this, the luteum function evaluation result is presented to the measurement subject. Accordingly, the user can easily obtain knowledge regarding her luteum function.

As has been made clear thus far, each of the various preferred embodiments of the present invention evaluates luteum function based on a basal body temperature measurement value and outputs the evaluation result. Accordingly, the user can easily obtain knowledge regarding her luteum function.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a block diagram illustrating the configuration of a luteum function evaluation system according to a preferred embodiment of the present invention.

FIG. 2 is a block diagram illustrating the configuration of a smartphone that is a part of the aforementioned system.

FIG. 3 is a block diagram illustrating the configuration of a server that is a part of the aforementioned system.

FIG. 4 is a block diagram illustrating the configuration of a thermometer that is a part of the aforementioned system.

FIG. 5 is a diagram illustrating an example of a luteum function evaluation result displayed in the smartphone that is part of the aforementioned system.

FIG. 6 is a diagram illustrating another example of a luteum function evaluation result displayed in the smartphone that is part of the aforementioned system.

FIG. 7 is a diagram illustrating an overall flow of operations performed by the server that is part of the aforementioned system.

FIG. 8A is a diagram illustrating a flow of operations, serving as part of the aforementioned overall flow of operations, involved in deriving an evaluation index.

FIG. 8B is a diagram illustrating a part of the aforementioned flow of operations involved in deriving an evaluation index.

FIG. 8C is a diagram illustrating a part of the aforementioned flow of operations involved in deriving an evaluation index.

FIG. 8D is a diagram illustrating a part of the aforementioned flow of operations involved in deriving an evaluation index.

FIG. 9A is a diagram illustrating a flow of operations, serving as part of the aforementioned overall flow of operations, involved in evaluating luteum function.

FIG. 9B is a diagram illustrating a part of the aforementioned flow of operations involved in evaluating luteum function.

FIG. 9C is a diagram illustrating a part of the aforementioned flow of operations involved in evaluating luteum function.

FIG. 9D is a diagram illustrating a part of the aforementioned flow of operations involved in evaluating luteum function.

FIG. 9E is a diagram illustrating a part of the aforementioned flow of operations involved in evaluating luteum function.

FIG. 9F is a diagram illustrating a part of the aforementioned flow of operations involved in evaluating luteum function.

FIG. 9G is a diagram illustrating a part of the aforementioned flow of operations involved in evaluating luteum function.

FIG. 10A is a diagram illustrating a flow of operations, serving as part of the aforementioned overall flow of operations, involved in evaluating items aside from luteum function.

FIG. 10B is a diagram illustrating a part of the aforementioned flow of operations involved in evaluating items aside from luteum function.

FIG. 11A is a diagram illustrating a part of the aforementioned flow of operations involved in schematic diagram rendering performed by the smartphone that is a part of the aforementioned system.

FIG. 11B is a diagram illustrating a part of the aforementioned flow of operations involved in the schematic diagram rendering.

FIG. 11C is a diagram illustrating a part of the aforementioned flow of operations involved in the schematic diagram rendering.

### DETAILED DESCRIPTION OF THE PREFERRED EMBODIMENTS

Hereinafter, preferred embodiments of the present invention will be described in detail with reference to the drawings.

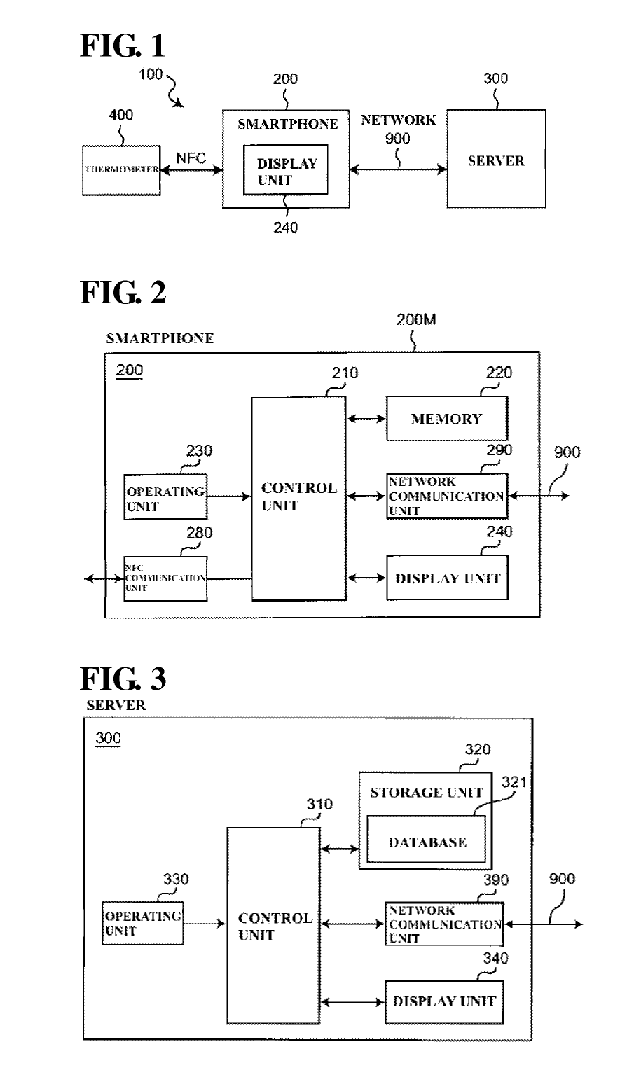

FIG. 1 is a diagram illustrating an example of a luteum function evaluation system according to a preferred embodiment of the present invention, indicated as a whole by reference numeral 100, in which a luteum function evaluation apparatus according to an aspect of various preferred embodiments of the present invention is configured as a server 300 on a network. The system 100 includes a smartphone 200 serving as a mobile terminal, the server 300 operating as the luteum function evaluation apparatus, and a thermometer 400. The smartphone 200 and the thermometer 400 are capable of communicating with each other through near-field communication (NFC). The smartphone 200 and the server 300 are capable of communicating with each other over a network 900.

As shown in FIG. 2, the smartphone 200 includes a main body 200M, as well as a control unit 210, a memory 220, an operating unit 230, a display unit 240, an NFC communication unit 280, and a network communication unit 290, which are provided in the main body 200M. The smartphone 200 is a commercially-available smartphone in which application software (computer programs) for executing processing that will be mentioned later has been installed.

The control unit 210 includes a central processing unit (CPU) as well as auxiliary circuitry thereof, controls the various units of the smartphone 200, and executes processes that will be mentioned later in accordance with programs and data stored in the memory 220. In other words, the control unit 210 processes data inputted through the operating unit 230 as well as the NFC communication unit 280 and the network communication unit 290, and stores the processed data in the memory 220, displays the processed data in the display unit 240, outputs the processed data from the NFC communication unit 280 and the network communication unit 290, or the like.

The memory 220 includes a random access memory (RAM) used as a work region required by the control unit 210 to execute programs, and a read-only memory (ROM) storing basic programs to be executed by the control unit 210. A semiconductor memory (a memory card, a solid-state drive (SSD)) or the like may be used as a storage medium in an auxiliary storage unit that complements a storage region in the memory 220.

The operating unit 230 is in this example configured of a touch panel provided on the display unit 240. Note, however, that another hardware-based operating device such as a keyboard may be included as well.

The display unit 240 includes a display screen (for example, a liquid-crystal display (LCD) or an electroluminescence (EL) display). The display unit 240 displays a predetermined image in the display screen under the control of the control unit 210.

The NFC communication unit 280 carries out near-field communication with the thermometer 400 when the thermometer 400 is near the smartphone 200, and receives data including a body temperature (a basal body temperature measurement value) and information indicating a measurement date/time from the thermometer 400.

The network communication unit 290 sends information from the control unit 210 to other apparatuses (in this example, the server 300) over the network 900, and receives information sent over the network 900 from another apparatus and passes the information to the control unit 210.

As shown in FIG. 3, the server 300 includes a control unit 310, a storage unit 320, an operating unit 330, a display unit 340, and a network communication unit 390. The server 300 is realized by installing computer programs (software) for executing processing, described later, in a generic computer device.

The control unit 310 includes a CPU (central processing unit) and other auxiliary circuits; the control unit 310 is configured or programmed to control the various elements of the server 300, execute predetermined processes in accordance with programs and data stored in the storage unit 320, process data inputted from the operating unit 330 and the network communication unit 390, store processed data in the storage unit 320, display processed data in the display unit 340, and output processed data from the network communication unit 390.

The storage unit 320 includes a RAM (random access memory) used as a work region required by the control unit 310 to execute programs, and a ROM (read-only memory) that stores basic programs to be executed by the control unit 310. A database 321 containing basal body temperature data sent from a user is provided in the storage unit 320. A magnetic disk (an HD (hard disk), an FD (flexible disk)), an optical disc (a CD (compact disc), a DVD (digital versatile disc), a BD (Blu-ray disc)), a magneto-optical disc (MO), or a semiconductor memory (a memory card, an SSD (solid state drive)), or the like may be used as a storage medium in an auxiliary storage apparatus to complement the storage region of the storage unit 320.

The operating unit 330 is in this example preferably including a keyboard and a mouse, and inputs operation signals indicating operations performed by a user (a server administrator, a user that is a measurement subject) to the control unit 310. Instead of or in addition to the keyboard and the mouse, the operating unit 330 may be configured of another operating device, such as a touch panel.

The display unit 340 includes a display screen (for example, a liquid-crystal display (LCD) or an electroluminescence (EL) display). The display unit 340 displays a predetermined image in the display panel under the control of the control unit 310.

The network communication unit 390 sends information from the control unit 310 to other apparatuses (in this example, the smartphone 200) over the network 900, and receives information sent over the network 900 from another apparatus and passes the information to the control unit 310.

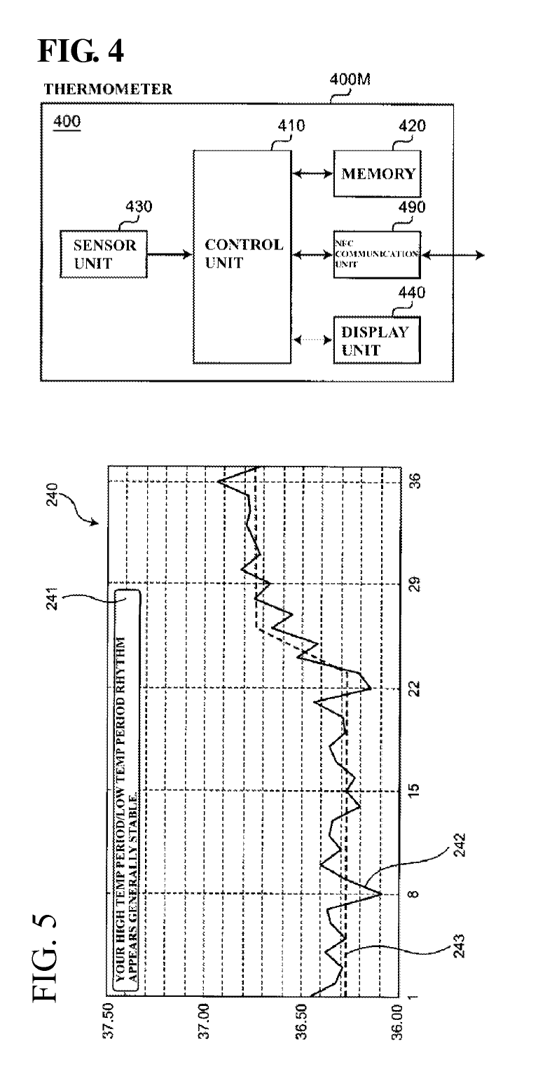

As shown in FIG. 4, the thermometer 400 preferably is in this case a commercially-available electronic womens' thermometer (MC-642L, manufactured by Omron Corporation), and includes a casing 400M, as well as a control unit 410, a memory 420, a sensor unit 430, a display unit 440, and an NFC communication unit 490 provided in the casing 400M.

The sensor unit 430 includes a temperature sensor, and measures and obtains a woman's body temperature (for example, a basal body temperature).

The memory 420 stores data of programs to control the thermometer 400, configuration data to configure various types of functions of the thermometer 400, body temperature measurement result data, and so on. In this example, the memory 420 can store several days' worth (for example, a maximum of 40 days' worth) of basal body temperature data along with the measurement date/times corresponding thereto. The memory 420 is also used as a working memory when programs are executed.

The control unit 410 includes a CPU (central processing unit), and is configured or programmed to control the memory 420, the display unit 440, and the NFC communication unit 490 in accordance with programs stored in the memory 420 to control the thermometer 400, based on detection signals from the sensor unit 430.

The display unit 440 includes a display screen that is in this example configured of a liquid-crystal display (LCD), and displays predetermined information in the display screen in accordance with signals received from the control unit 410.

The NFC communication unit 490 carries out near-field communication with the smartphone 200 when the thermometer 400 is near the smartphone 200, and sends data indicating the basal body temperature measurement value and the measurement date/time to the smartphone 200.

The system 100 preferably is used as follows.

A user (measurement subject) measures her basal body temperature every day for a plurality of days within a cycle, such as a menstrual cycle, using the thermometer 400. In this example, the basal body temperature measurement value data is stored in the memory 420 of the thermometer 400 along with the measurement date/time when that basal body temperature was measured.

Then, each time the user brings the thermometer 400 near the smartphone 200, the NFC communication unit 490 (a first communication unit) of the thermometer 400 and the NFC communication unit 280 (a second communication unit) of the smartphone 200 communicate with each other, and the data held in the thermometer 400, such as the basal body temperature measurement value, is sent to the smartphone 200. Through this, the smartphone 200 obtains, from the thermometer 400, data indicating the basal body temperature measurement value of the measurement subject, and the measurement date/times thereof, for each day in a plurality of days.

Note that the thermometer 400 and the smartphone 200 preferably communicates every day, and in this example, the communication preferably is carried out periodically within a range that ensures a maximum number of measurement results that can be stored by the thermometer 400 will not be exceeded.

Note that in the case where the measurement subject has measured the body temperature using a thermometer that does not include the NFC communication unit 490, the user can also manually input the data indicating the basal body temperature measurement value and the measurement date/times by operating the operating unit 230 of the smartphone 200.

The user also inputs information regarding her current menstrual period, such as a menstruation start day, a menstruation end day, and the menstrual cycle, using the operating unit 230 of the smartphone 200. The user may also input information regarding a previous menstrual period (a menstrual period in which the day before the menstruation start day for the current menstrual period serves as the final day).

Note that the smartphone 200 or the server 300 preferably determines the information regarding the current menstrual period by estimating the menstrual cycle (the menstruation start day and the menstruation end day) through a known method, based on the basal body temperature measurement value. In the case where the user has only inputted the menstruation start day (or the menstruation end day), the stated estimation preferably takes into consideration the menstruation start day (or menstruation end day) inputted by the user and estimate the menstruation end day (or menstruation start day) and the menstrual cycle. In such a case, the woman who is the user does not need to input information such as the menstruation start day, the menstruation end day, and the menstrual cycle through the operating unit 230, which eliminates the burden of such input.

Next, the user operates the operating unit 230 of the smartphone 200, and causes the smartphone 200 to send various types of information from the network communication unit 290 to the server 300 over the network 900. Here, the various types of information include the basal body temperature measurement values and measurement date/time data (basal body temperature data) obtained by the smartphone 200 from the thermometer 400, and data indicating the information regarding the current menstrual period. The various types of information preferably also include data indicating information regarding the previous menstrual period.

The server 300 receives, via the network communication unit 390, the basal body temperature data and the data indicating the information regarding the current menstrual period from the network 900. At this time, the server 300 preferably also receives the data indicating the information regarding the previous menstrual period. Note that the server 300 preferably estimates the menstrual cycle (the menstruation start day and the menstruation end day) through a known method based on the basal body temperature measurement value included in the received basal body temperature data and preferably then determines the information regarding the current and previous menstrual periods. Furthermore, the user may input the information regarding the current and previous menstrual periods into the server 300 by operating a device aside from the smartphone 200 (the server 300 itself, a personal computer (not shown) connected to the network 900, or the like).

Under the control of the control unit 310 operating as a body temperature obtainment unit, the server 300 stores, in the database 321 of the storage unit 320, the basal body temperature data received by the network communication unit 390 from the smartphone 200 over the network 900.

Note that the server 300 preferably checks whether the basal body temperature measurement values included in the received basal body temperature data contain an abnormal value resulting from a fever caused by some illness affecting the measurement subject, an abnormal value resulting from a measurement error or the like, and so on, and exclude detected abnormal values from the basal body temperature data. An estimated value corresponding to the excluded body temperature measurement value preferably is then interpolated using other basal body temperature measurement values next to the measurement days of the excluded body temperature measurement values, and preferably stores the estimated value in the database 321. The server 300 preferably also checks the received basal body temperature data, and in the case where a day of no measurement has been confirmed, an estimated value corresponding to the basal body temperature measurement value for the day of no measurement preferably then is interpolated using other basal body temperature measurement values next to the day of no measurement; the estimated value preferably then is stored in the database 321.

Furthermore, under the control of the control unit 310 operating as a menstrual cycle obtainment unit, the server 300 stores, in the database 321 of the storage unit 320, data indicating the information regarding the current menstrual period, the previous menstrual period, or the like (the menstruation start day, the menstruation end day, and so on) received by the network communication unit 390 from the smartphone 200 over the network 900. Note that in the case where the server 300 determines the information regarding the current menstrual period, the previous menstrual period, or the like through estimation, the control unit 310 that operates as the menstrual cycle obtainment unit stores, in the database 321, a result of the estimation, which is performed by the control unit 310 operating as a menstrual cycle estimation unit.

Next, the control unit 310 of the server 300, operating as an evaluation unit, evaluates the luteum function of the user, who is the measurement subject, based on the basal body temperature measurement value measured over a plurality of days in the current menstrual cycle (a span of time from the menstruation start day to the menstruation end day). Specifically, the control unit 310 operating as the evaluation unit calculates an evaluation index for evaluating the luteum function from the plurality of basal body temperature measurement values and evaluates whether the luteum function is normal or insufficient based on the evaluation index. Accordingly, the control unit 310 operating as the evaluation unit is capable of at least a two-phase evaluation, indicating whether the luteum function is normal or insufficient, based on a plurality of basal body temperature measurement values.

Furthermore, in the case where the luteum function is abnormal, the control unit 310 operating as the evaluation unit is capable of estimating whether or not ovulation is occurring based on the evaluation index. Accordingly, the control unit 310 operating as the evaluation unit is also capable of at least a three-phase evaluation, indicating that the luteum function is normal, or that the luteum function is insufficient and ovulation is (was) occurring, or that the luteum function is insufficient and ovulation is not (was not) occurring, based on the plurality of basal body temperature measurement values.

In addition to the stated luteum function evaluation, the control unit 310 operating as the evaluation unit preferably also is configured or programmed to evaluate the measurement subject's menstrual cycle, a length of a follicular phase, and so on.

When the evaluation performed by the evaluation unit ends, the server 300 outputs an evaluation result from the network communication unit 390 to the smartphone 200 over the network 900, under the control of the control unit 310 operating as an output unit. The evaluation result includes, for example, the evaluation of the luteum function, the menstrual cycle, the length of the follicular phase, and so on, and preferably further includes a comment determined based on the evaluations collectively, data such as images representing the evaluation result as graphics, audio, and so on. The evaluation result preferably also includes information regarding a plurality of evaluation indices derived from the basal body temperature measurement values during the evaluation.

Note that the server 300 preferably displays the evaluation result in its own display unit 340 under the control of the control unit 310 operating as the output unit. Alternatively, the server 300 may output the evaluation result as audio through its own audio synthesis unit (not shown) under the control of the control unit 310 operating as the output unit. The method by which the server 300 outputs the evaluation result includes sending the evaluation result over the network 900, displaying an image using the display unit 340, outputting audio through the audio synthesis unit, and so on, and is not limited thereto.

The smartphone 200 receives data indicating the evaluation result sent from the server 300 over the network 900 using the network communication unit 290.

The smartphone 200 stores the received data indicating the evaluation result in the memory 220. When the user then instructs the smartphone 200 to display the evaluation result by operating the operating unit 230 of the smartphone 200, the smartphone 200 displays the evaluation result in the display unit 240 under the control of the control unit 210 operating as a notifying unit.

Note that the smartphone 200 preferably outputs the evaluation result as audio through its own audio synthesis unit (not shown) under the control of the control unit 210 operating as the notifying unit. The method by which the smartphone 200 notifies the user of the evaluation result includes displaying an image using the display unit 240, outputting audio through the audio synthesis unit, and so on, and is not limited thereto.

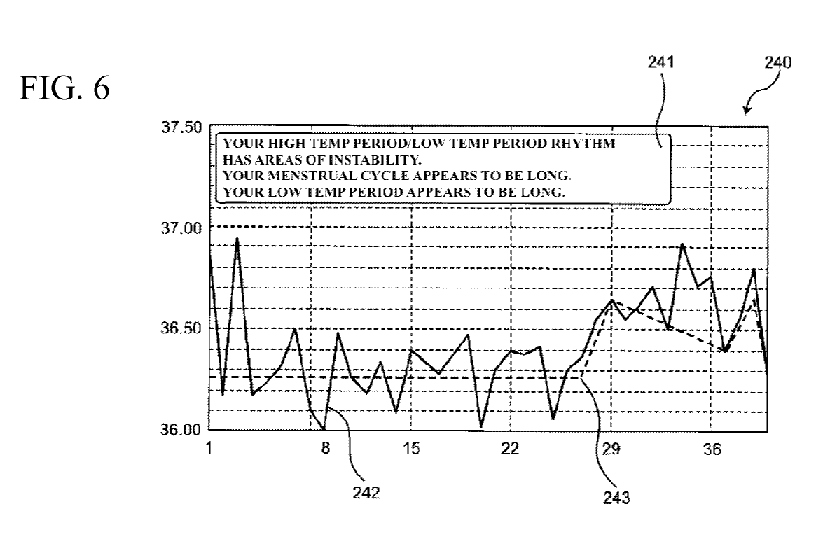

Specifically, the smartphone 200 displays the evaluation result as shown in FIGS. 5 and 6 under the control of the control unit 210 operating as the notifying unit. FIG. 5 illustrates an example of a result display in the case where the luteum function has been evaluated as normal, and FIG. 6 illustrates an example of a result display in the case where the luteum function has been evaluated as insufficient.

As shown in FIGS. 5 and 6, an evaluation comment 241 determined based on the collective evaluations of the luteum function, the menstrual cycle, and the length of the follicular phase is displayed in the display unit 240 of the smartphone 200, along with a basal body temperature curve 242 and a basal body temperature curve schematic diagram 243 generated by extracting a characteristic of the basal body temperature curve. The image data of the schematic diagram 243 is superimposed on the basal body temperature curve 242 and displayed as the schematic diagram 243 in the display unit 240 by the control unit 210 of the smartphone 200 operating as a rendering unit and generating a schematic diagram image of the basal body temperature curve based on the evaluation result for luteum function and the like. The schematic diagram 243 is displayed in order to help the user more intuitively understand the characteristics of her own basal body temperature curve.

In the examples shown in FIGS. 5 and 6, the evaluation result is communicated to the user as the evaluation comment 241, but the method for communicating the evaluation result is not limited thereto. The evaluation result preferably is displayed and communicated to the user as separate evaluations of luteum function, menstrual cycle, and length of the follicular phase.

In this manner, the system 100 according to a preferred embodiment of the present invention preferably evaluates luteum function and the like based on the measurement subject's (user's) basal body temperature measurement values and can then notify the user of the evaluation result. According to the system 100, the user can easily, and without involving a third party, obtain knowledge regarding her own luteum function, which thus far has required a doctor's examination. In addition to notifying the user of the evaluation result (example: the evaluation comment 241), the system 100 also presents the basal body temperature curve schematic diagram 243 to the user. Accordingly, the user is presented with the underlying data for the evaluation result in a format that can be intuitively understood, which facilitates the user's understanding of her own physical condition. This provides a further advantage in that the user is encouraged to proactively select appropriate actions, such as being examined by a doctor, in the case where the evaluation result includes content that is not positive.

Operations performed by the server 300 that operates as the luteum function evaluation apparatus will be described in detail hereinafter with reference to the flowcharts in the appended drawings.

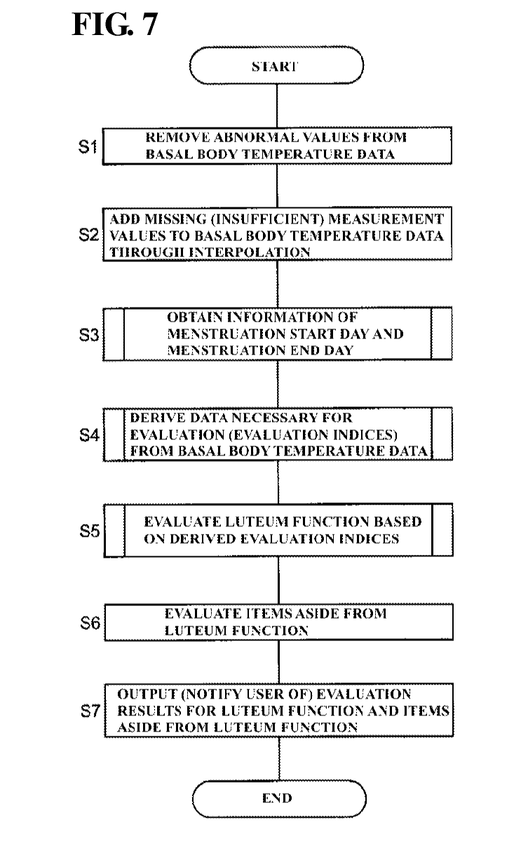

First, an overview of the flow of server operations will be described with reference to FIG. 7.

Here, it is assumed that the server 300 already holds, in the storage unit 320, the basal body temperature data and the data indicating the information regarding the current menstrual period of the measurement subject.

In step S1, the control unit 310 of the server 300 operates as the body temperature obtainment unit, checking whether the basal body temperature measurement values contain an abnormal value resulting from a fever caused by some illness affecting the measurement subject, an abnormal value resulting from a measurement error or the like, and so on, and excludes detected abnormal values from the basal body temperature data.

In step S2, the control unit 310 generates an estimated value corresponding to the excluded body temperature measurement value through interpolation using other basal body temperature measurement values next to the measurement days of the excluded body temperature measurement values, and reconstructs the basal body temperature data. In addition, the control unit 310 of the server 300 generates the estimated value corresponding to the basal body temperature measurement value for the day of no measurement through interpolation using other basal body temperature measurement values next to the day of no measurement, and reconstructs the basal body temperature data.

In step S3, the control unit 310 of the server 300 specifies the menstruation start day and the menstruation end day of the current menstrual period based on the data indicating the information regarding the current menstrual period, by operating as the menstrual cycle obtainment unit.

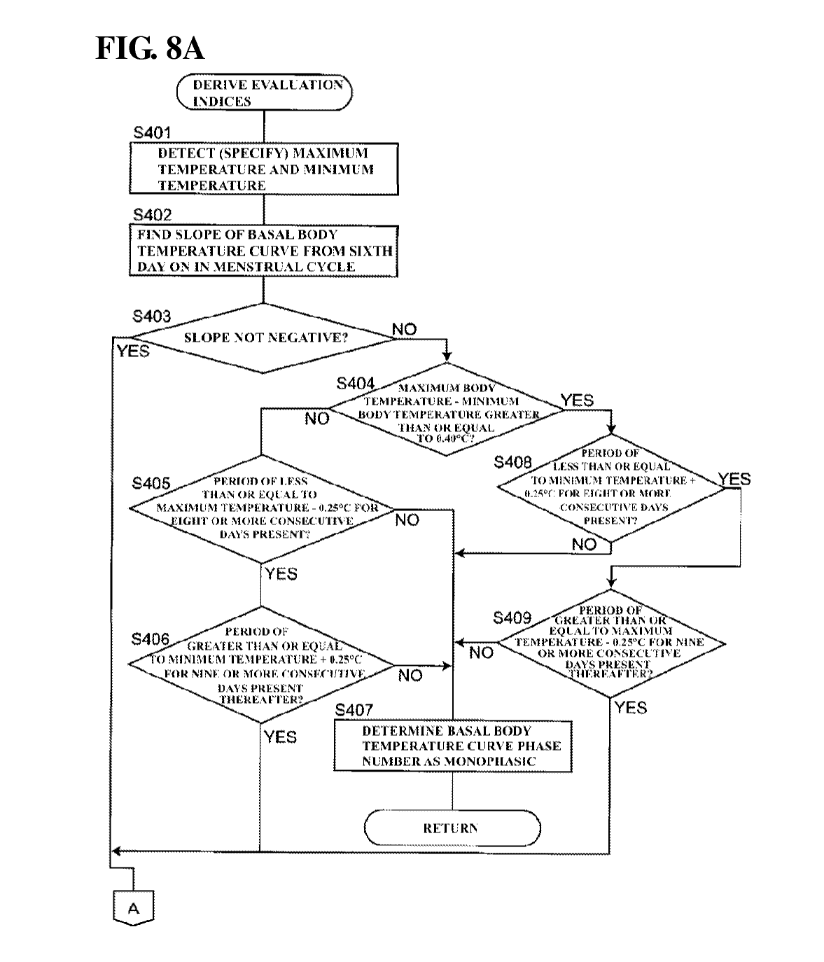

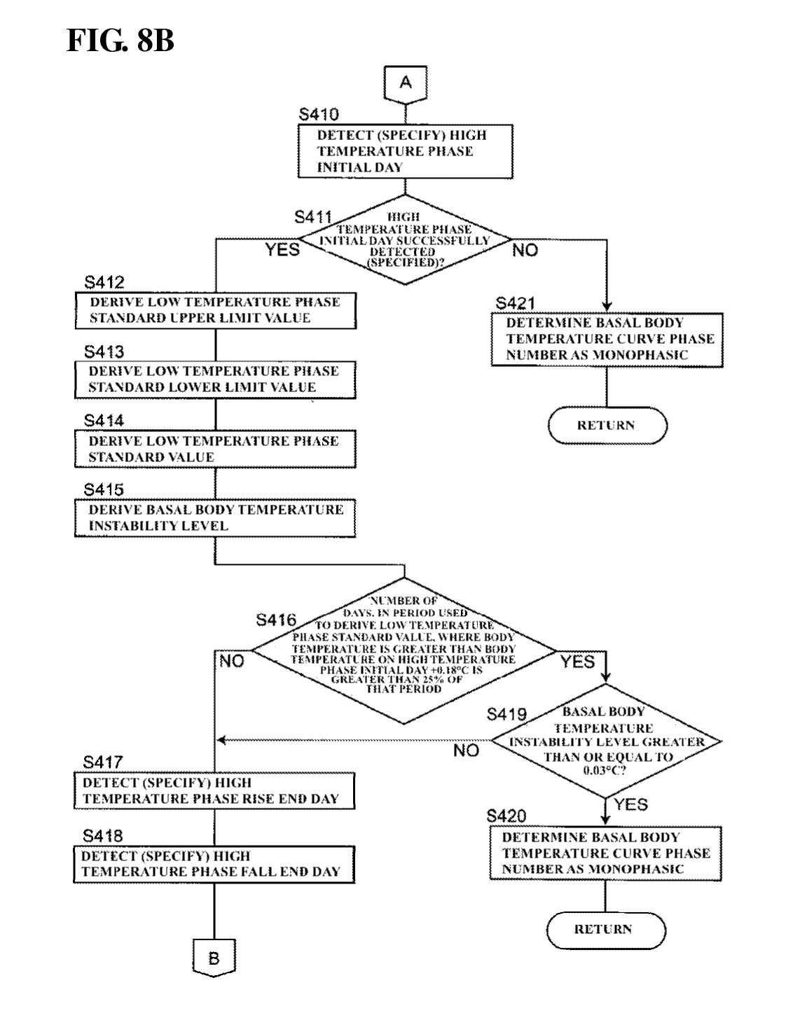

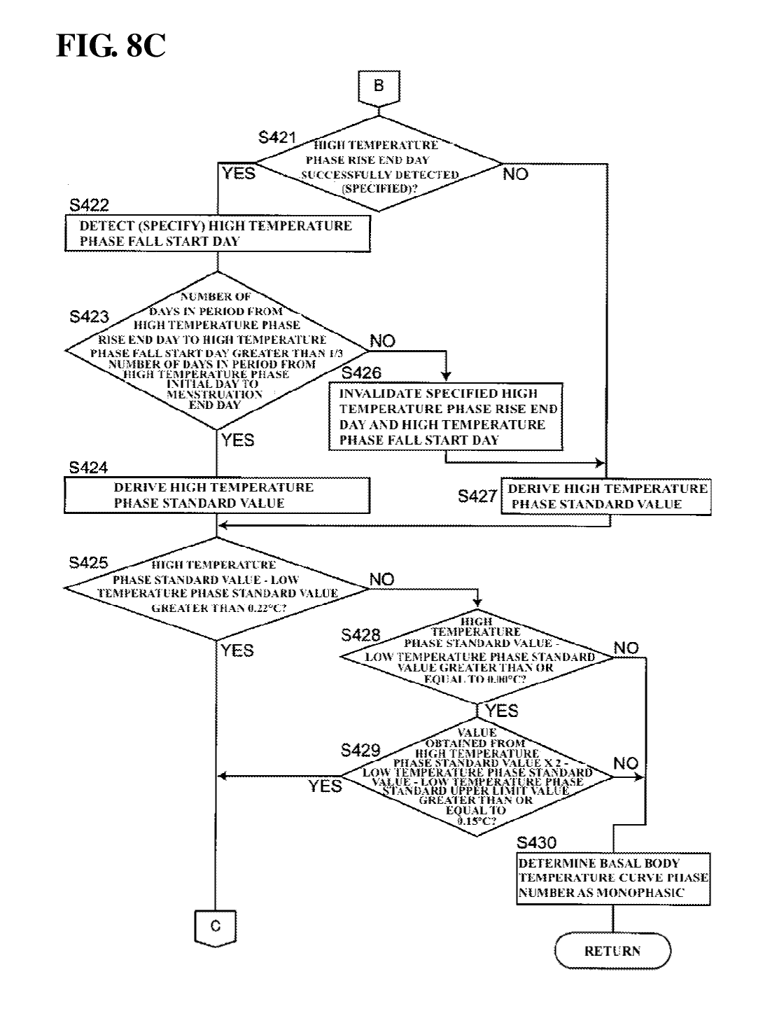

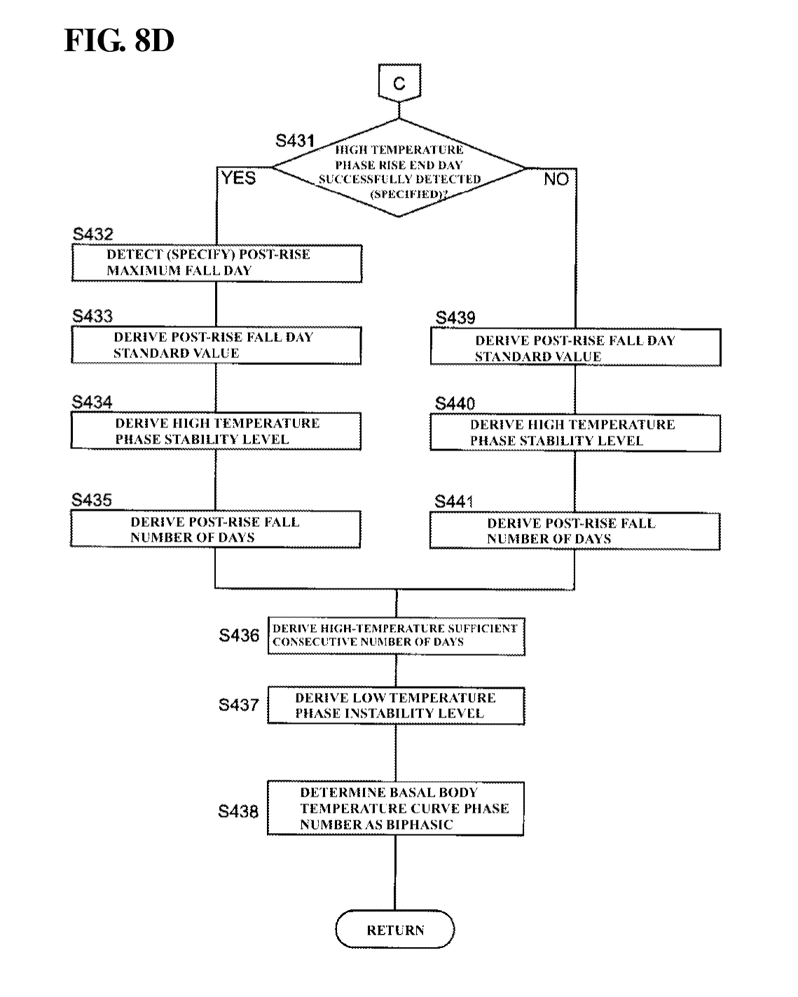

In step S4, the control unit 310 of the server 300 derives a plurality of evaluation indices, mentioned later, based on the plurality of basal body temperature measurement values contained in the basal body temperature data, by operating as the evaluation unit. The evaluation indices and the operations for deriving the evaluation indices performed in step S4 will be described in detail later with reference to FIGS. 8A through 8D.

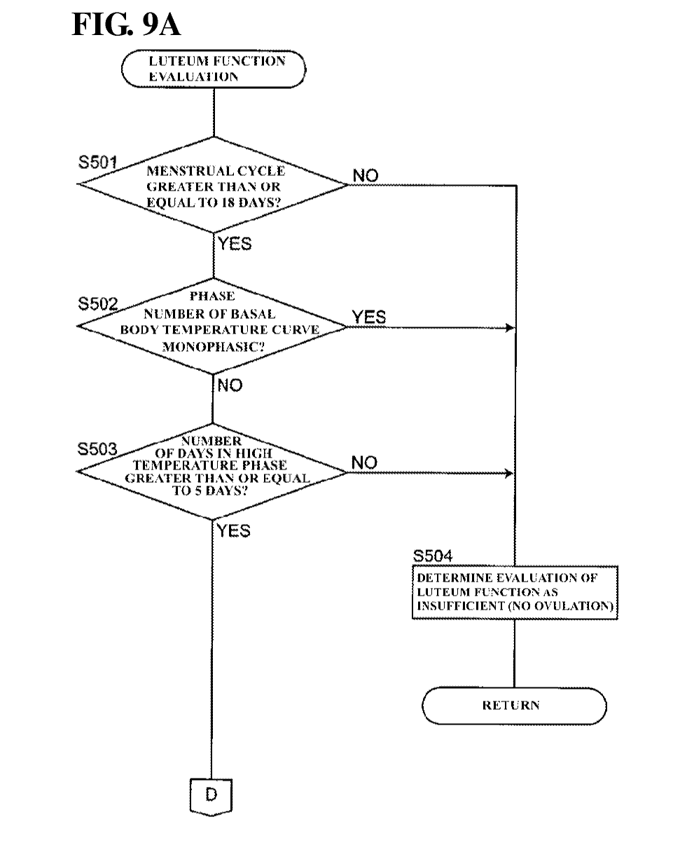

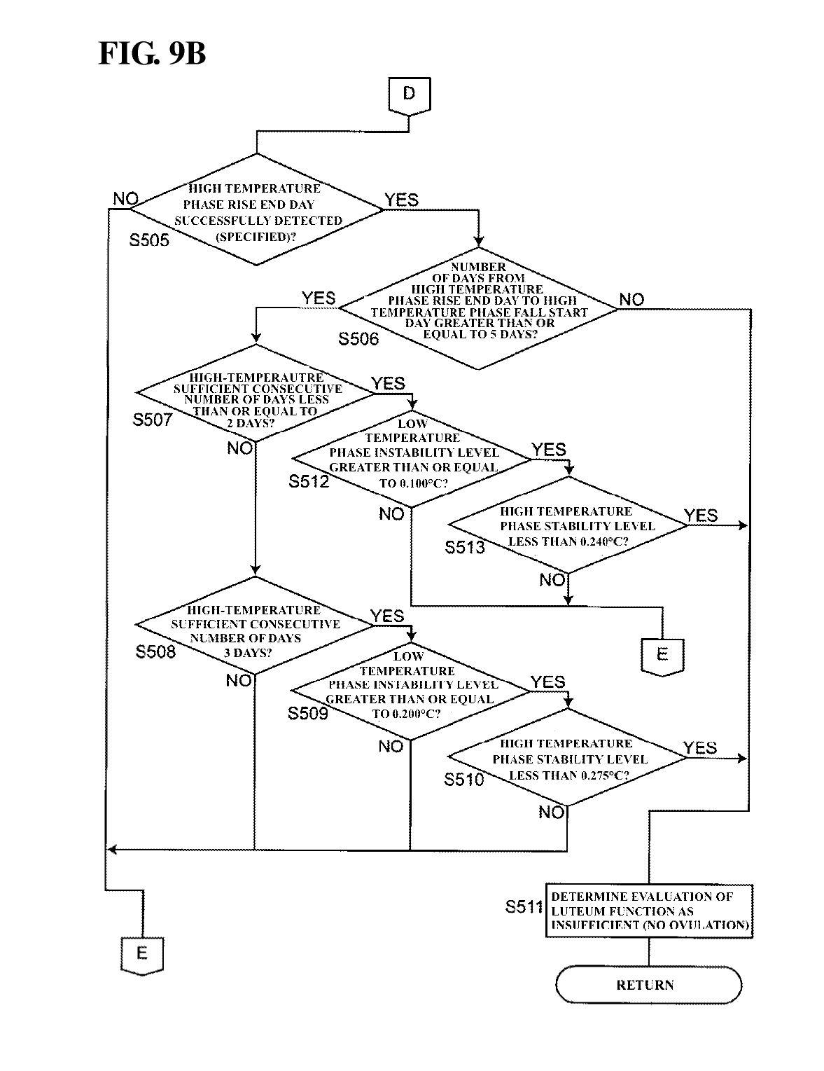

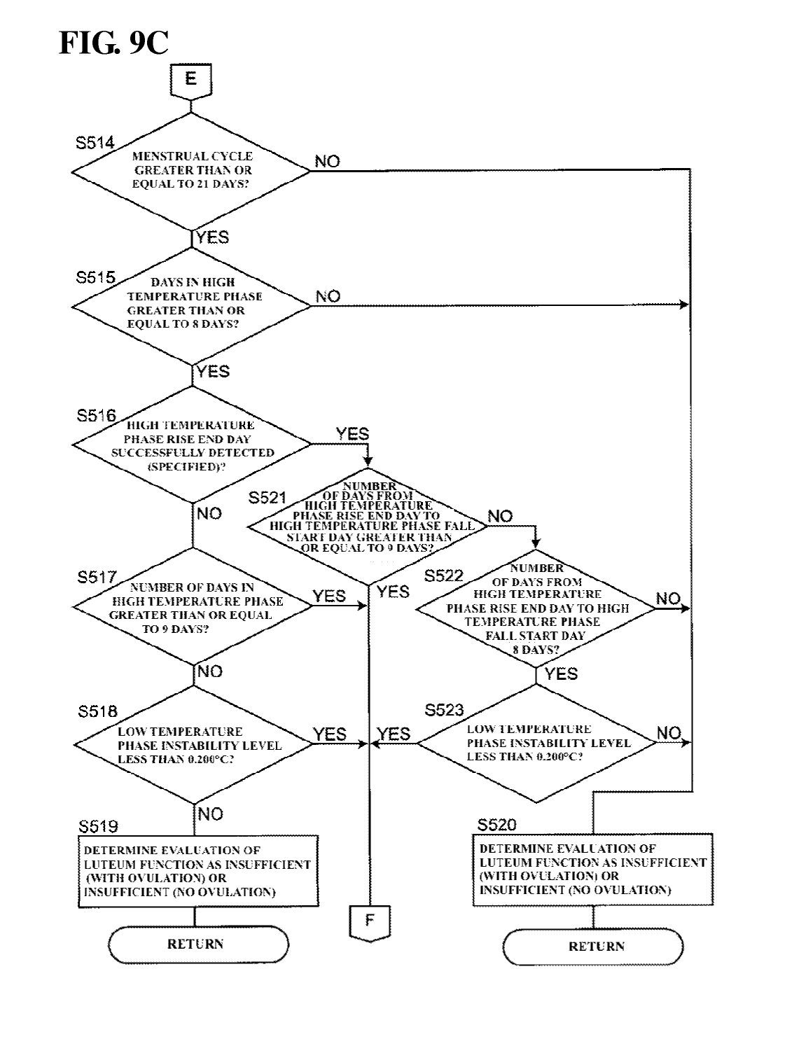

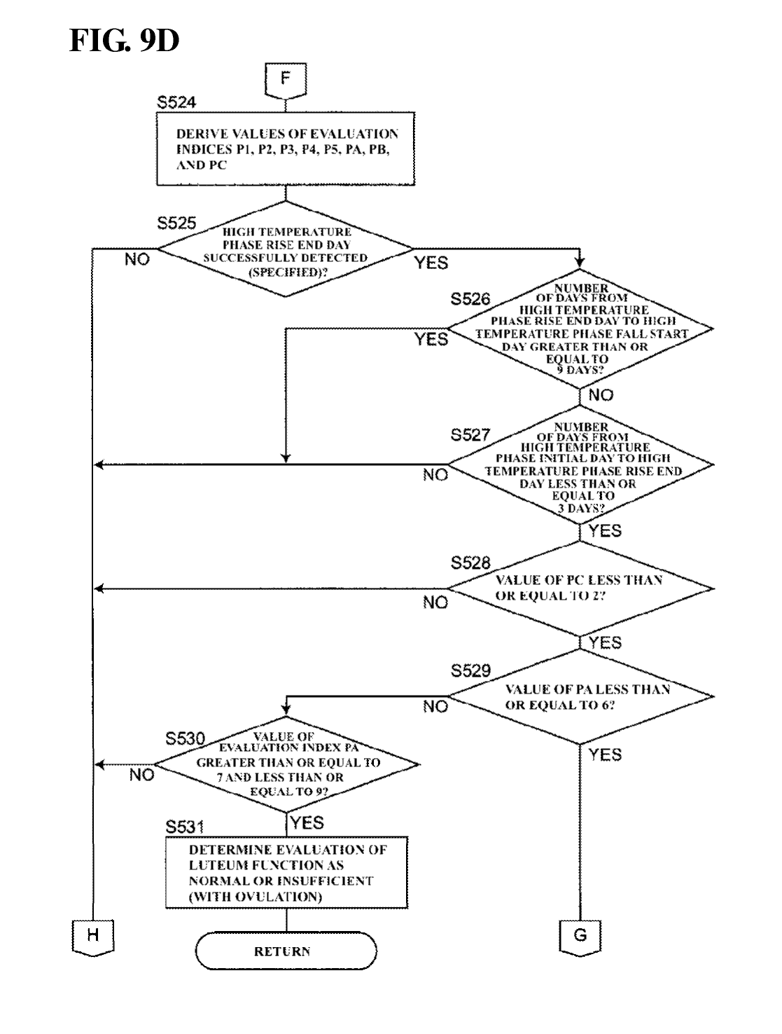

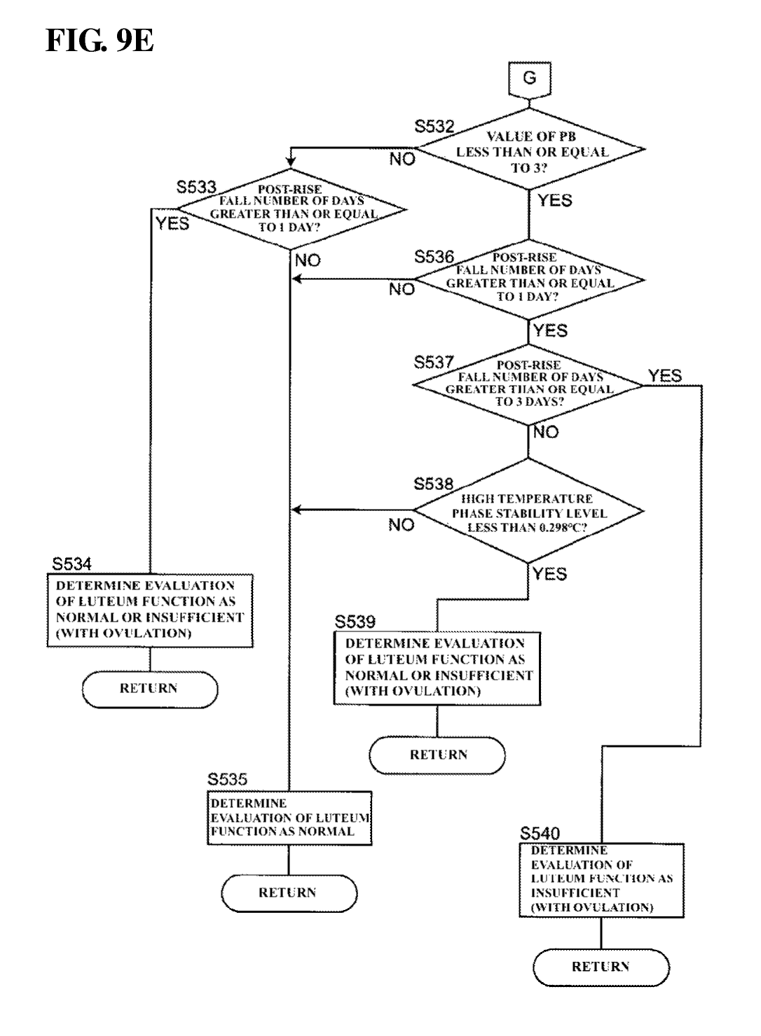

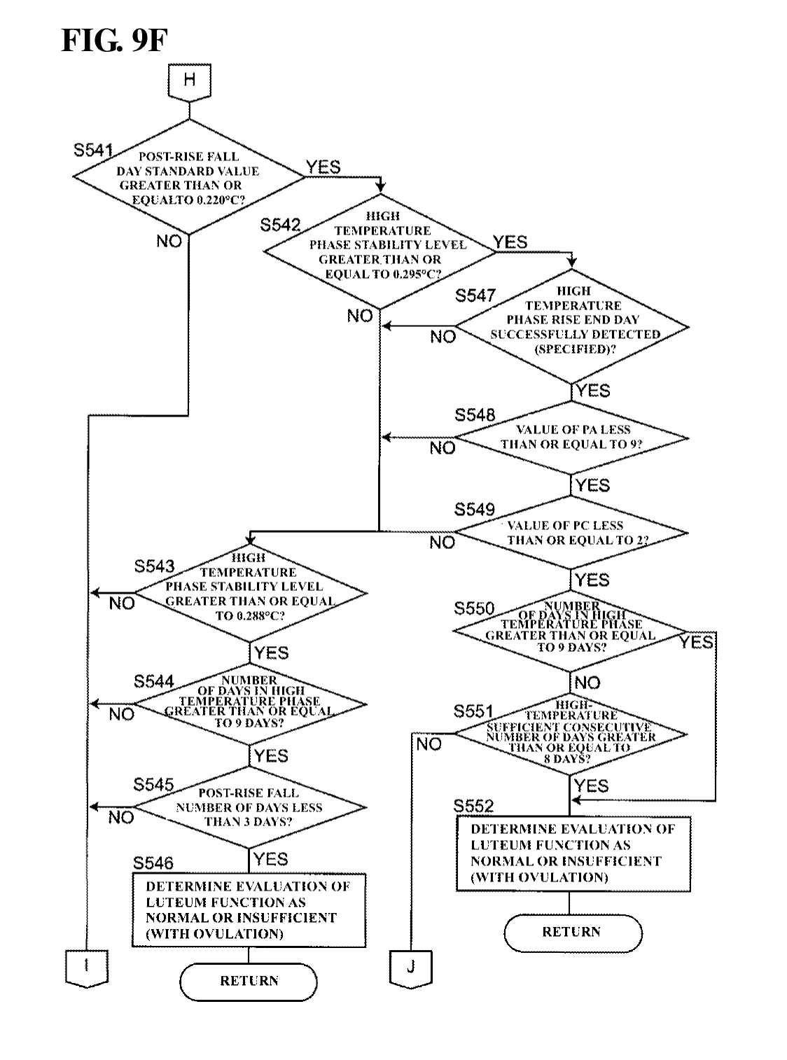

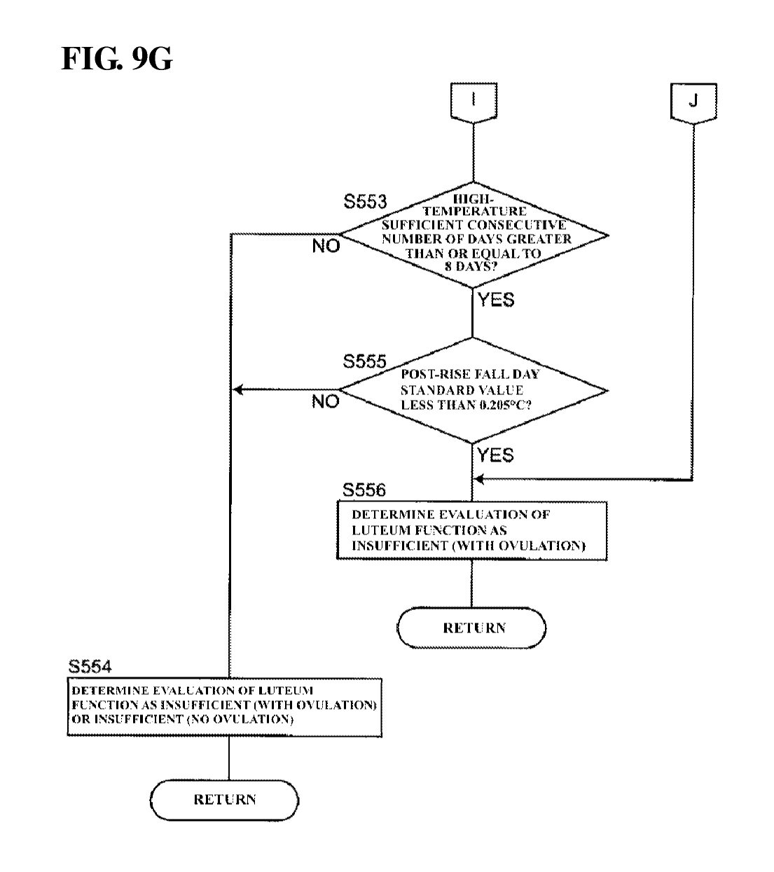

In step S5, the control unit 310 of the server 300 evaluates luteum function based on the plurality of evaluation indices derived based on the basal body temperature measurement value, by operating as the evaluation unit. The operations for evaluating luteum function performed in step S5 will be described in detail later with reference to FIGS. 9A through 9G.

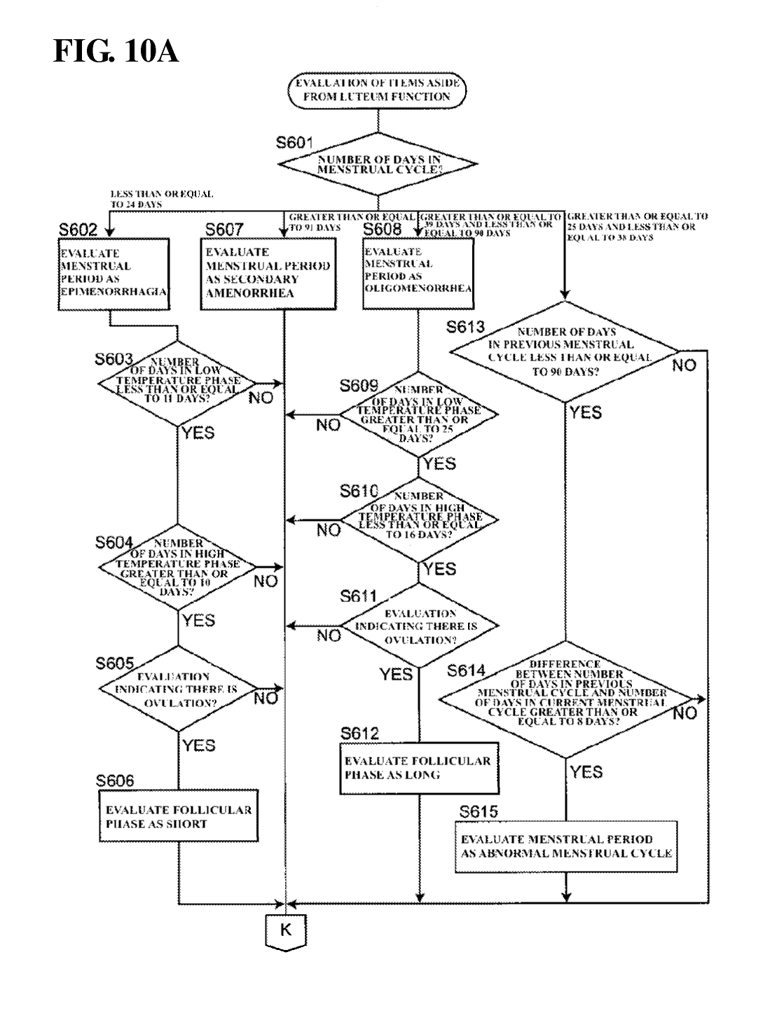

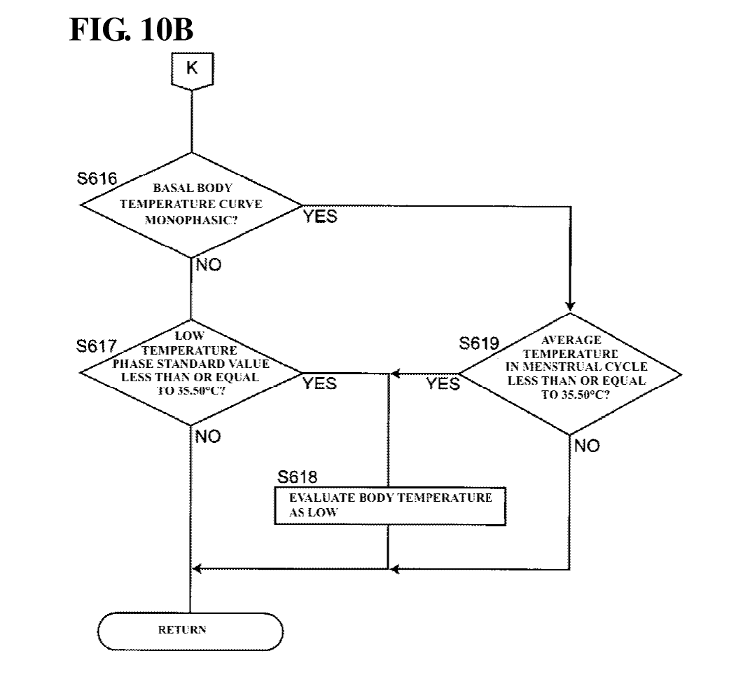

In step S6, the control unit 310 of the server 300 evaluates items aside from luteum function based on the plurality of evaluation indices derived based on the basal body temperature measurement value, by operating as the evaluation unit. The evaluation of items aside from luteum function includes, for example, evaluating the menstrual cycle, evaluating the length of the follicular phase, or the like. The operations for evaluating the items aside from luteum function performed in step S6 will be described in detail later with reference to FIGS. 10A and 10B.

In step S7, the control unit 310 of the server 300 outputs the evaluation results for luteum function and the items aside from luteum function in a predetermined format, by operating as the output unit. According to the system 100, the evaluation results are sent to the smartphone 200.

Next, the evaluation indices and the operations for deriving the evaluation indices performed in step S4 will be described in detail with reference to FIGS. 8A through 8D.

Referring to FIG. 8A, in step S401, the control unit 310 operating as the evaluation unit (referred to simply as the “evaluation unit” hereinafter) specifies a maximum value and a minimum value of the basal body temperature measurement values in the current menstrual period as “maximum temperature” and “minimum temperature” evaluation indices, respectively.

In step S402, the evaluation unit finds the slope of a basal body temperature curve from the sixth day on (evaluation index “slope sixth day on”) of the current menstrual period (hereinafter, this will be referred to simply as the “menstrual cycle” when there is no risk of unclearness). Note that the menstrual cycle is a period that takes the menstruation start day as a first day and the menstruation end day as a final day.

The “slope sixth day on” referred to here is the slope of an approximated straight line obtained when the basal body temperature measurement values from the sixth day of the menstrual cycle to the menstruation end day are approximated using a straight line, through the least-squares method, for example.

In step S403, the evaluation unit determines whether or not the index “slope sixth day on” found in step S402 is not negative (is zero or positive). When the index “slope sixth day on” is not negative (“YES”), the process moves to step S410. However, when the index “slope sixth day on” is negative (“NO”), the process moves to step S404.

In step S404, the evaluation unit determines whether or not a difference between the maximum temperature and the minimum temperature (maximum temperature−minimum temperature) is greater than or equal to 0.40° C. When the difference is greater than or equal to 0.40° C. (“YES”), the process moves to step S408. However, when the difference is less than 0.40° C. (“NO”), the process moves to step S405.

In step S405, the evaluation unit determines, based on the plurality of basal body temperature measurement values in the menstrual cycle, whether or not there is a period in which the basal body temperature measurement value is less than or equal to the maximum temperature−0.25° C. for eight or more consecutive days. In the case where it has been determined that such a period is present (“YES”), the process moves to step S406. However, in the case where it has been determined that such a period is not present (“NO”), the process moves to step S407.

In step S406, the evaluation unit determines, based on the plurality of basal body temperature measurement values in the menstrual cycle, whether or not there is a period in which the basal body temperature measurement value is greater than or equal to the minimum temperature+0.25° C. for nine or more consecutive days following the period determined to be present in step S405. In the case where it has been determined that such a period is present (“YES”), the process moves to step S410. However, in the case where it has been determined that such a period is not present (“NO”), the process moves to step S407.

In step S407, the evaluation unit sets an evaluation index “phase number” to monophasic. Note that the evaluation index “phase number” is an index regarding whether the basal body temperature curve is biphasic, including a high temperature phase and a low temperature phase, or is not biphasic (that is, is monophasic).

In step S408, the evaluation unit determines, based on the plurality of basal body temperature measurement values in the menstrual cycle, whether or not there is a period in which the basal body temperature measurement value is less than or equal to the minimum temperature+0.25° C. for eight or more consecutive days. In the case where it has been determined that such a period is present (“YES”), the process moves to step S409. However, in the case where it has been determined that such a period is not present (“NO”), the process moves to step S407 (the evaluation index “phase number” is set to monophasic).

In step S409, the evaluation unit determines, based on the plurality of basal body temperature measurement values in the menstrual cycle, whether or not there is a period in which the basal body temperature measurement value is greater than or equal to the maximum temperature−0.25° C. for nine or more consecutive days following the period determined to be present in step S408. In the case where it has been determined that such a period is present (“YES”), the process moves to step S410. However, in the case where it has been determined that such a period is not present (“NO”), the process moves to step S407 (the evaluation index “phase number” is set to monophasic).

Referring to FIG. 8B, in step S410, the evaluation unit specifies an evaluation index “high temperature phase initial day”.

Here, the “high temperature phase initial day” refers to a day after the fifth day in the menstrual cycle, and is a day where the basal body temperature measurement value measured on that day is greater than +0.10° C. of an average value of the basal body temperature measurement values from the first day of the menstrual cycle. However, in the case where more than one day meets this condition, the day nearest to 14 days before the menstruation end day is taken as the “high temperature phase initial day”.

In step S411, the evaluation unit determines whether or not the evaluation index “high temperature phase initial day” has been successfully specified in step S410 (that is, whether or not a day that meets the stated condition is present). In the case where the evaluation index “high temperature phase initial day” has been specified in step S410 (“YES”), the process moves to step S412. However, in the case where the evaluation index “high temperature phase initial day” has not been specified in step S410 (“NO”), the process moves to step S421.

In step S412, the evaluation unit derives an evaluation index “low temperature phase standard upper limit value”.

Here, the “low temperature phase standard upper limit value” is a maximum value in the basal body temperature measurement values from the seventh day of the menstrual cycle to a low temperature phase end day (the day before the high temperature phase initial day).

In step S413, the evaluation unit derives an evaluation index “low temperature phase standard lower limit value”.

Here, the “low temperature phase standard lower limit value” is a minimum value in the basal body temperature measurement values from the seventh day of the menstrual cycle to the low temperature phase end day (the day before the high temperature phase initial day).

In step S414, the evaluation unit derives an evaluation index “low temperature phase standard value”.

Here, the “low temperature phase standard value” is a value determined as follows:

low temperature phase standard value=(low temperature phase standard upper limit value+low temperature phase standard lower limit value)/2

In step S415, the evaluation unit derives an evaluation index “basal body temperature instability level”.

Here, the “basal body temperature instability level” is a total sum of the basal body temperature measurement values in the days from the seventh day of the menstrual cycle to the low temperature phase end day, indicated as follows:

basal body temperature instability level=(ΣMax(basal body temperature measurement value−(basal body temperature measurement value of high temperature phase initial day+0.18° C.),0))/(range of number of days for low temperature phase standard value calculation)

The range of number of days for low temperature phase standard value calculation is the number of days from the seventh day of the menstrual cycle to the low temperature phase end day.

In step S416, the evaluation unit determines whether or not the number of days where the basal body temperature measurement value is greater than the basal body temperature measurement value of the high temperature phase initial day+0.18° C. is greater than 25% of the range of number of days for low temperature phase standard value calculation, in a low temperature phase standard value calculation range (a period from the seventh day of the menstrual cycle to the low temperature phase end day). In the case where it has been determined that the stated number is greater than 25% of the range of number of days for low temperature phase standard value calculation (“YES”), the process moves to step S419. In the case where it has been determined that the stated number is not greater than 25% of the range of number of days for low temperature phase standard value calculation (“NO”), the process moves to step S417.

In step S417, the evaluation unit specifies an evaluation index “high temperature phase rise end day”.

Here, the “high temperature phase rise end day” refers to a day where the basal body temperature has risen sufficiently, and specifically refers to a day following the high temperature phase initial day, on which the basal body temperature measurement value is greater than or equal to the low temperature phase standard value+0.30° C., and is the first day where the basal body temperature measurement value is greater than or equal to the low temperature phase standard upper limit value.

In step S418, the evaluation unit specifies an evaluation index “high temperature phase fall end day”.

Here, the “high temperature phase fall end day” refers to the last day where the basal body temperature is comparatively high, and specifically, refers to a day following the high temperature phase initial day, on which the basal body temperature measurement value is greater than or equal to the low temperature phase standard upper limit value, and that is the final day.

In the case where the process has advanced from step S416 to step S419, in step S419, the evaluation unit determines whether or not the evaluation index “basal body temperature instability level” is greater than or equal to 0.03° C. In the case where it has been determined that the basal body temperature instability level is greater than or equal to 0.03° C. (“YES”), the process moves to step S420. However, in the case where it has been determined that the basal body temperature instability level is less than 0.03° C. (“NO”), the process moves to step S417.

In step S420, the evaluation unit sets the evaluation index “phase number” to monophasic.

Meanwhile, in the case where the process has advanced from step S411 to step S421, in step S421, the evaluation unit sets the evaluation index “phase number” to monophasic.

Referring to FIG. 8C, in step S421, the evaluation unit determines whether or not the evaluation index “high temperature phase rise end day” has been successfully specified in step S417 (that is, whether or not a day that meets the stated condition is present). In the case where the evaluation index “high temperature phase rise end day” has been specified in step S417 (“YES”), the process moves to step S422. However, in the case where the evaluation index “high temperature phase rise end day” has not been specified in step S417 (“NO”), the process moves to step S427.

In step S422, the evaluation unit specifies an evaluation index “high temperature phase fall start day”.

Here, the “high temperature phase fall start day” refers to a day following the high temperature phase initial day, on which the basal body temperature measurement value for that day is greater than or equal to the low temperature phase standard value+0.30° C., and is the last day where the basal body temperature measurement value is greater than or equal to the low temperature phase standard upper limit value.

In step S423, the evaluation unit determines whether or not the number of days in a period from the high temperature phase rise end day to the high temperature phase fall start day is greater than one-third the number of days in a period from the high temperature phase initial day to the menstruation end day. In the case where it has been determined that the number of days in the period from the high temperature phase rise end day to the high temperature phase fall start day is greater than one-third the number of days in the period from the high temperature phase initial day to the menstruation end day (“YES”), the process moves to step S424. However, in the case where it has been determined that the number is not greater (“NO”), the process moves to step S426.

In step S424, the evaluation unit derives an evaluation index “high temperature phase standard value”.

In the derivation process carried out in step S424, an average value of the basal body temperature measurement values from the high temperature phase rise end day to the high temperature phase fall start day is derived and used as the “high temperature phase standard value”.

Meanwhile, in the case where the process has advanced from step S423 to step S426, in step S426, the evaluation unit invalidates the specified “high temperature phase rise end day” and “high temperature phase fall start day”.

Then, in step S427, the evaluation unit derives the evaluation index “high temperature phase standard value”.

However, the derivation process carried out in step S427 differs from that carried out in step S424 in that an average value of the basal body temperature measurement values from the high temperature phase initial day to the high temperature phase fall end day is derived and used as the “high temperature phase standard value”.

In both step S424 and step S427, the evaluation index “high temperature phase standard value” is derived as an index indicating the average value of the basal body temperature measurement values in the high temperature phase.

In step S425, the evaluation unit determines whether or not a difference between the high temperature phase standard value and the low temperature phase standard value (high temperature phase standard value−low temperature phase standard value) is greater than 0.22° C. In the case where it has been determined that the difference is greater than 0.22° C. (“YES”), the process moves to step S431. However, in the case where it has been determined that the difference is less than or equal to 0.22° C. (“NO”), the process moves to step S428.

In step S428, the evaluation unit determines whether or not a difference between the high temperature phase standard value and the low temperature phase standard value (high temperature phase standard value−low temperature phase standard value) is greater than or equal to 0.00° C. In the case where it has been determined that the difference is greater than or equal to 0.00° C. (“YES”), the process moves to step S429. However, in the case where it has been determined that the difference is less than 0.00° C. (“NO”), the process moves to step S430.

In step S429, the evaluation unit determines whether or not a value obtained by subtracting the low temperature phase standard value and the low temperature phase standard upper limit value from a value twice the high temperature phase standard value is greater than or equal to 0.15° C. In the case where it has been determined that the value resulting from the subtraction is greater than or equal to 0.15° C. (“YES”), the process moves to step S431. However, in the case where it has been determined that the value resulting from the subtraction is less than 0.15° C. (“NO”), the process moves to step S430.

In step S430, the evaluation unit sets the evaluation index “phase number” to monophasic.

Referring to FIG. 8D, in step S431, the evaluation unit determines whether or not the evaluation index “high temperature phase rise end day” has been successfully specified (has been specified and has not been invalidated). In the case where it has been determined that the evaluation index “high temperature phase rise end day” has been specified (“YES”), the process moves to step S432. However, in the case where it has been determined that the evaluation index “high temperature phase rise end day” has not been specified (“NO”), the process moves to step S439.

In step S432, the evaluation unit specifies an evaluation index “post-rise maximum fall day”.

Here, the “post-rise maximum fall day” refers to a day in the high temperature phase where the amount of a fall from the previous day's basal body temperature measurement value is greatest, and specifically refers to a day in which the basal body temperature measurement value is the lowest among the following candidates:
candidate 1: a day, in a period from the day after the high temperature phase rise end day to the day two days before the high temperature phase fall start day, in which the basal body temperature measurement value on that day is less than or equal to the high temperature phase standard value+0.10° C.; and
candidate 2: a day, of the day after the high temperature phase rise end day and the high temperature phase fall start day, in which the basal body temperature measurement value on that day is greater than the low temperature phase standard value+0.20° C. and less than or equal to the high temperature phase standard value+0.20° C.

In step S433, the evaluation unit derives an evaluation index “post-rise fall day standard value”.

In the derivation process performed in step S433, the “post-rise fall day standard value” is a total sum of the basal body temperature measurement values in the days from the high temperature phase rise end day to the high temperature phase fall start day, indicated as follows:

post-rise fall day standard value=(ΣMax(Min(basal body temperature measurement value, low temperature phase standard value+0.30° C.)−low temperature phase standard value, 0))/(number of days in post-rise fall day standard value calculation range in which basal body temperature measurement value is less than low temperature phase standard value+0.30° C.)

The post-rise fall day standard value calculation range is a period from the high temperature phase rise end day to the high temperature phase fall start day. However, in the case where the numerator in the above equation is 0° C. and the denominator is one day, the post-rise fall day standard value is set to 0.300° C.

In step S434, the evaluation unit derives an evaluation index “high temperature phase stability level”.

In the derivation process performed in step S434, the “high temperature phase stability level” is a total sum of the basal body temperature measurement values in the days from the high temperature phase rise end day to the high temperature phase fall start day, indicated as follows:

high temperature phase stability level=(ΣMax(Min(basal body temperature measurement value, low temperature phase standard value+0.30° C.)−low temperature phase standard value, 0))/(number of days in high temperature phase stability level calculation range)

The number of days in high temperature phase stability level calculation range is the number of days from the high temperature phase rise end day to the high temperature phase fall start day.

In step S435, the evaluation unit derives an evaluation index “post-rise fall number of days”.

In the derivation process performed in step S435, the “post-rise fall number of days” refers to the number of days, from the high temperature phase rise end day to the high temperature phase fall start day, in which the basal body temperature measurement values on those days are less than the low temperature phase standard value+0.30° C.

Meanwhile, in the case where the process has advanced from step S431 to step S439, in step S439, the evaluation unit derives the evaluation index “post-rise fall day standard value”.

However, the derivation process performed in step S439 differs from that performed in step S433 in that the “post-rise fall day standard value” is a total sum of the basal body temperature measurement values in the days from the high temperature phase initial day to a high temperature phase final day, indicated as follows:

post-rise fall day standard value=(ΣMax(Min(basal body temperature measurement value, low temperature phase standard value+0.30° C.)−low temperature phase standard value, 0))/(number of days in post-rise fall day standard value calculation range in which basal body temperature measurement value is less than low temperature phase standard value+0.30° C.)

The post-rise fall day standard value calculation range is a period from the high temperature phase initial day to the high temperature phase final day. However, in the case where the numerator in the above equation is 0° C. and the denominator is one day, the post-rise fall day standard value is set to 0.300° C.

In both step S433 and step S439, the evaluation index “post-rise fall day standard value” is derived as an index indicating the average value of only the basal body temperature measurement values lower than the low temperature phase standard value+0.30° C., of the basal body temperature measurement values in the high temperature phase.

Meanwhile, in the case where the process has advanced from step S431 to step S439, in step S440, the evaluation unit derives the evaluation index “high temperature phase stability level”.

However, the derivation process performed in step S440 differs from that performed in step S434 in that the “high temperature phase stability level” is a total sum of the basal body temperature measurement values in the days from the high temperature phase initial day to the high temperature phase final day, indicated as follows:

high temperature phase stability level=(ΣMax(Min(basal body temperature measurement value, low temperature phase standard value+0.30° C.)−low temperature phase standard value, 0))/(number of days in high temperature phase stability level calculation range)

The number of days in high temperature phase stability level calculation range is the number of days from the high temperature phase initial day to the high temperature phase final day.

In both step S434 and step S440, the evaluation index “high temperature phase stability level” is derived as an index indicating the stability of the basal body temperature measurement value throughout the high temperature phase.

Meanwhile, in the case where the process has advanced from step S431 to step S439, in step S441, the evaluation unit derives the evaluation index “post-rise fall number of days”.

However, the derivation process performed in step S441 differs from that performed in step S435 in that the “post-rise fall number of days” is the number of days, from the high temperature phase initial day to the high temperature phase final day, in which the basal body temperature measurement values on those days are less than the low temperature phase standard value+0.30° C.

In both step S435 and step S441, the evaluation index “post-rise fall number of days” is derived as an index indicating the number of days in the high temperature phase in which the basal body temperature measurement value is less than the low temperature phase standard value+0.30° C.

Next, in step S436, the evaluation unit derives an evaluation index “high-temperature sufficient consecutive number of days”.

Here, the “high-temperature sufficient consecutive number of days” is a partial period configured of days following the high temperature phase initial day, and is the number of days in the longest period of consecutive days in which the basal body temperature measurement value is greater than or equal to Max(low temperature phase standard value+0.20° C., low temperature phase standard upper limit value) (higher temperature of low temperature phase standard value+0.20° C. and low temperature phase standard upper limit value).

In other words, the index “high-temperature sufficient consecutive number of days” is derived as an index indicating the number of consecutive days in the low temperature phase in which the basal body temperature measurement value is higher than a predetermined temperature.

Next, in step S437, the evaluation unit derives an evaluation index “low temperature phase instability level”.

Here, the “low temperature phase instability level” is an index indicating a level of fluctuation in the basal body temperature measurement value during the low temperature phase, and specifically is a total sum of the basal body temperature measurement values in the days from the seventh day of the menstrual cycle to the low temperature phase end day, indicated as follows:

low temperature phase instability level=(absolute value of difference between basal body temperature measurement values from two consecutive days in which a temperature difference between the basal body temperature measurement values of the two consecutive days is greater than or equal to 0.15° C.)/(range of number of days for low temperature phase standard value calculation−1)

The range of number of days for low temperature phase standard value calculation is the number of days from the seventh day of the menstrual cycle to the low temperature phase end day.

In step S438, the evaluation unit sets the evaluation index “phase number” to biphasic.

Through the processing performed in step S4 as described above, the evaluation unit derives the following as the evaluation indices: the maximum temperature, the minimum temperature, the slope sixth day on, the phase number, the high temperature phase initial day, the high temperature phase rise end day, the high temperature phase fall start day, the high temperature phase fall end day, the high temperature phase standard value, the low temperature phase standard value, the low temperature phase standard upper limit value, the low temperature phase standard lower limit value, the basal body temperature instability level, the low temperature phase instability level, the high temperature phase stability level, the post-rise maximum fall day, the post-rise fall day standard value, the post-rise fall number of days, and the high-temperature sufficient consecutive number of days.

In the processing performed in step S5 and step S6, which will be described hereinafter, the evaluation unit determines the luteum function evaluation and the evaluation of items aside from luteum function using several of the plurality of evaluation indices derived in step S4.

The operations for evaluating luteum function will be described in detail next with reference to FIGS. 9A through 9G.

Referring to FIG. 9A, in step S501, the evaluation unit determines whether or not the menstrual cycle is greater than or equal to 18 days. When the menstrual cycle is greater than or equal to 18 days (“YES”), the process moves to step S502. However, when the menstrual cycle is less than 18 days (“NO”), the process moves to step S504.

In step S502, the evaluation unit determines whether or not the phase number of the basal body temperature curve is monophasic. When the basal body temperature curve is biphasic (“NO”), the process moves to step S503. However, when the basal body temperature curve is monophasic (“YES”), the process moves to step S504.

In step S503, the evaluation unit determines whether or not the number of days in the high temperature phase in the menstrual cycle is greater than or equal to five days. When the number of days in the high temperature phase is greater than or equal to five days (“YES”), the process moves to step S505. However, when the number of days in the high temperature phase is less than five days (“NO”), the process moves to step S504.

In step S504, the evaluation unit determines the evaluation of luteum function to be “insufficient (no ovulation)”.

Referring to FIG. 9B, in step S505, the evaluation unit determines whether or not the evaluation index “high temperature phase rise end day” has been successfully specified in step S4. In the case where the evaluation index “high temperature phase rise end day” has been specified in step S4 (“YES”), the process moves to step S506. However, in the case where the evaluation index “high temperature phase rise end day” has not been specified in step S4 (“NO”), the process moves to step S514.

In step S506, the evaluation unit determines whether or not the number of days from the high temperature phase rise end day to the high temperature phase fall start day is greater than or equal to five days. In the case where that number of days is greater than or equal to five days (“YES”), the process moves to step S507. However, in the case where that number of days is less than five days (“NO”), the process moves to step S511.

In step S507, the evaluation unit determines whether or not the high-temperature sufficient consecutive number of days is less than or equal to two days. In the case where that number of days is less than or equal to two days (“YES”), the process moves to step S512. However, in the case where that number of days is greater than two days (“NO”), the process moves to step S508.

In step S508, the evaluation unit determines whether or not the high-temperature sufficient consecutive number of days is three days. In the case where that number of days is three days (“YES”), the process moves to step S509. However, in the case where that number of days is not three days (“NO”), the process moves to step S514.

In step S509, the evaluation unit determines whether or not the low temperature phase instability level is greater than or equal to 0.200° C. In the case where the low temperature phase instability level is greater than or equal to 0.200° C. (“YES”), the process moves to step S510. However, in the case where that instability level is less than 0.200° C. (“NO”), the process moves to step S514.

In step S510, the evaluation unit determines whether or not the high temperature phase stability level is less than 0.275° C. In the case where the high temperature phase stability level is less than 0.275° C. (“YES”), the process moves to step S511. However, in the case where that stability level is greater than or equal to 0.275° C. (“NO”), the process moves to step S514.

In step S511, the evaluation unit determines the evaluation of luteum function to be “insufficient (no ovulation)”.

Meanwhile, in step S512, the evaluation unit determines whether or not the low temperature phase instability level is greater than or equal to 0.100° C. In the case where the low temperature phase instability level is greater than or equal to 0.100° C. (“YES”), the process moves to step S513. However, in the case where that instability level is less than 0.100° C. (“NO”), the process moves to step S514.

In step S513, the evaluation unit determines whether or not the high temperature phase stability level is less than 0.240° C. In the case where the high temperature phase stability level is less than 0.240° C. (“YES”), the process moves to step S511. However, in the case where that stability level is greater than or equal to 0.240° C. (“NO”), the process moves to step S514.

Referring to FIG. 9C, in step S514, the evaluation unit determines whether or not the menstrual cycle is greater than or equal to 21 days. When the menstrual cycle is greater than or equal to 21 days (“YES”), the process moves to step S515. However, when the menstrual cycle is less than 21 days (“NO”), the process moves to step S520.

In step S515, the evaluation unit determines whether or not the number of days in the high temperature phase in the menstrual cycle is greater than or equal to eight days. When the number of days in the high temperature phase is greater than or equal to eight days (“YES”), the process moves to step S516. However, when the number of days in the high temperature phase is less than eight days (“NO”), the process moves to step S520.

In step S516, the evaluation unit determines whether or not the evaluation index “high temperature phase rise end day” has been successfully specified in step S4. In the case where the evaluation index “high temperature phase rise end day” has been specified in step S4 (“YES”), the process moves to step S521. However, in the case where the evaluation index “high temperature phase rise end day” has not been specified in step S4 (“NO”), the process moves to step S517.

In step S517, the evaluation unit determines whether or not the number of days in the high temperature phase in the menstrual cycle is greater than or equal to nine days. When the number of days in the high temperature phase is greater than or equal to nine days (“YES”), the process moves to step S524. However, when the number of days in the high temperature phase is less than nine days (“NO”), the process moves to step S518.

In step S518, the evaluation unit determines whether or not the low temperature phase instability level is less than 0.200° C. In the case where the low temperature phase instability level is less than 0.200° C. (“YES”), the process moves to step S524. However, in the case where that instability level is greater than or equal to 0.200° C. (“NO”), the process moves to step S519.

In step S519, the evaluation unit determines the evaluation of luteum function to be “insufficient (with ovulation) or insufficient (no ovulation)”.

In step S521, the evaluation unit determines whether or not the number of days from the high temperature phase rise end day to the high temperature phase fall start day is greater than or equal to nine days. In the case where that number of days is greater than or equal to nine days (“YES”), the process moves to step S524. However, in the case where that number of days is less than nine days (“NO”), the process moves to step S522.

In step S522, the evaluation unit determines whether or not the number of days from the high temperature phase rise end day to the high temperature phase fall start day is eight days. In the case where that number of days is eight days (“YES”), the process moves to step S523. However, in the case where that number of days is not eight days (“NO”), the process moves to step S520.

In step S523, the evaluation unit determines whether or not the low temperature phase instability level is less than 0.200° C. In the case where the low temperature phase instability level is less than 0.200° C. (“YES”), the process moves to step S524. However, in the case where that instability level is greater than or equal to 0.200° C. (“NO”), the process moves to step S520.

Like step S519, in step S520, the evaluation unit determines the evaluation of luteum function to be “insufficient (with ovulation) or insufficient (no ovulation)”.

Referring to FIG. 9D, in step S524, the evaluation unit derives secondary evaluation indices (P1, P2, P3, P4, P5, PA, PB, and PC), which are derived from the evaluation indices found in step S4.

Although the process for deriving the secondary evaluation indices will be described hereinafter, the processes performed to derive the evaluation indices P1, P2, P3, P4, and P5 will be described first. P1 to P5 can each take on a value of 1, 2, or 3. The evaluation indices P1 to P5 are each indices that indicate the quality of luteum function from different standpoints, and in each index, a lower value indicates better luteum function.

The index P1 is a value returned by the following function f1.

The function f1 is a function that:

returns a value of 1 when the high temperature phase standard value is greater than or equal to Max(low temperature phase standard value+0.30° C., (low temperature phase standard value−35.25° C.)×0.9+35.70° C.);

returns a value of 2 when the above condition is not met and the high temperature phase standard value is greater than or equal to Max (low temperature phase standard value+0.25° C., (low temperature phase standard value−35.25° C.)×0.9+35.65° C.); and

returns a value of 3 when neither of the above two conditions are met.

The index P2 is a value returned by the following function f2.

The function f2 is a function that:

returns a value of 1 when the high temperature phase standard value is greater than or equal to Max(low temperature phase standard upper limit value+0.20° C., (low temperature phase standard upper limit value−35.00° C.)×(6/7)+35.40° C.);

returns a value of 2 when the above condition is not met and the high temperature phase standard value is greater than or equal to Max(low temperature phase standard upper limit value+0.05° C., (low temperature phase standard upper limit value−35.05° C.)×(6/7)+35.35° C.); and

returns a value of 3 when neither of the above two conditions are met.

The index P3 is a value returned by the following function f3.

The function f3 is a function that:

returns a value of 1 when the basal body temperature measurement value on the post-rise maximum fall day is greater than or equal to Max(low temperature phase standard value+0.40° C., (low temperature phase standard value−35.20° C.)×(2/3)+35.90° C.);

returns a value of 2 when the above condition is not met and the basal body temperature measurement value on the post-rise maximum fall day is greater than or equal to Max(low temperature phase standard value+0.24° C., (low temperature phase standard value−35.12° C.)×(7/8)+35.53° C.); and

returns a value of 3 when neither of the above two conditions are met.

The index P4 is a value returned by the following function f4.

The function f4 is a function that:

returns a value of 1 when the basal body temperature measurement value on the post-rise maximum fall day is greater than or equal to a value obtained by adding 0.16° C. to the low temperature phase standard upper limit value;

returns a value of 2 when the above condition is not met and the basal body temperature measurement value on the post-rise maximum fall day is greater than or equal to a value obtained by subtracting 0.05° C. from the low temperature phase standard upper limit value; and

returns a value of 3 when neither of the above two conditions are met.

The index P5 is a value returned by the following function f5.

The function f5 is a function that:

returns a value of 1 when the basal body temperature measurement value on the post-rise maximum fall day is greater than or equal to a value obtained by subtracting 0.16° C. from the high temperature phase standard value;

returns a value of 2 when the above condition is not met and the basal body temperature measurement value on the post-rise maximum fall day is greater than or equal to Min(Max(high temperature phase standard value−0.42° C., (high temperature phase standard value−35.00° C.)×0.5+35.45° C.), high temperature phase standard value−0.26° C.); and

returns a value of 3 when neither of the above two conditions are met.

Next, the processes for deriving the evaluation indices PA, PB, and PC will be described. The evaluation indices PA to PC are each indices that indicate the quality of luteum function from different standpoints, and in each index, a lower value indicates better luteum function.

The index PA is derived as PA=P1+P2+P3+P4+P5.

The index PB is derived as PB=P3+P4+P5.

The index PC is derived as the maximum value of P1 to P5 (PC=Max(P1, P2, P3, P4, P5)).

Next, in step S525, the evaluation unit determines whether or not the evaluation index “high temperature phase rise end day” has been successfully specified in step S4. In the case where the evaluation index “high temperature phase rise end day” has been specified in step S4 (“YES”), the process moves to step S526. However, in the case where the evaluation index “high temperature phase rise end day” has not been specified in step S4 (“NO”), the process moves to step S541.

In step S526, the evaluation unit determines whether or not the number of days from the high temperature phase rise end day to the high temperature phase fall start day is greater than or equal to nine days. In the case where that number of days is greater than or equal to nine days (“YES”), the process moves to step S541. However, in the case where that number of days is less than nine days (“NO”), the process moves to step S527.

In step S527, the evaluation unit determines whether or not the number of days from the high temperature phase initial day to the high temperature phase rise end day is less than or equal to three days. In the case where that number of days is less than or equal to three days (“YES”), the process moves to step S528. However, in the case where that number of days is greater than three days (“NO”), the process moves to step S541.

In step S528, the evaluation unit determines whether or not the value of the secondary evaluation index PC derived in step S524 is less than or equal to 2. In the case where the value of PC is less than or equal to 2 (“YES”), the process moves to step S529. However, in the case where that value is greater than 2 (“NO”), the process moves to step S541.

In step S529, the evaluation unit determines whether or not the value of the secondary evaluation index PA derived in step S524 is less than or equal to 6. In the case where the value of PA is less than or equal to 6 (“YES”), the process moves to step S532. However, in the case where that value is greater than 6 (“NO”), the process moves to step S530.

In step S530, the evaluation unit determines whether or not the value of the secondary evaluation index PA derived in step S524 is greater than or equal to 7 and less than or equal to 9. In the case where the value of PA is greater than or equal to 7 and less than or equal to 9 (“YES”), the process moves to step S531. However, in the case where that value is greater than 9 (“NO”), the process moves to step S541.

In step S531, the evaluation unit determines the evaluation of luteum function to be “normal or insufficient (with ovulation)”.

Referring to FIG. 9E, in step S532, the evaluation unit determines whether or not the value of the secondary evaluation index PB derived in step S524 is less than or equal to 3. In the case where the value of PB is less than or equal to 3 (“YES”), the process moves to step S536. However, in the case where that value is greater than 3 (“NO”), the process moves to step S533.

In step S533, the evaluation unit determines whether or not the post-rise fall number of days is greater than or equal to one day. In the case where the post-rise fall number of days is greater than or equal to one day (“YES”), the process moves to step S534. However, in the case where the post-rise fall number of days is less than one day (“NO”), the process moves to step S535.

In step S534, the evaluation unit determines the evaluation of luteum function to be “normal or insufficient (with ovulation)”.

Meanwhile, in step S535, the evaluation unit determines the evaluation of luteum function to be “normal”.

Meanwhile, in step S536, the evaluation unit determines whether or not the post-rise fall number of days is greater than or equal to one day. In the case where the post-rise fall number of days is greater than or equal to one day (“YES”), the process moves to step S537. However, in the case where the post-rise fall number of days is less than one day (“NO”), the process moves to step S535.

In step S537, the evaluation unit determines whether or not the post-rise fall number of days is greater than or equal to three days. In the case where the post-rise fall number of days is greater than or equal to three days (“YES”), the process moves to step S540. However, in the case where the post-rise fall number of days is less than three days (“NO”), the process moves to step S538.

In step S538, the evaluation unit determines whether or not the high temperature phase stability level is less than 0.298° C. In the case where the high temperature phase stability level is less than 0.298° C. (“YES”), the process moves to step S539. However, in the case where that stability level is greater than or equal to 0.298° C. (“NO”), the process moves to step S535.

In step S539, the evaluation unit determines the evaluation of luteum function to be “normal or insufficient (with ovulation)”.

Meanwhile, in step S540, the evaluation unit determines the evaluation of luteum function to be “insufficient (with ovulation)”.

Referring to FIG. 9F, in step S541, the evaluation unit determines whether or not the post-rise fall day standard value is greater than or equal to 0.220° C. In the case where the post-rise fall day standard value is greater than or equal to 0.220° C. (“YES”), the process moves to step S542. However, in the case where that standard value is less than 0.220° C. (“NO”), the process moves to step S553.

In step S542, the evaluation unit determines whether or not the high temperature phase stability level is greater than or equal to 0.295° C. In the case where the high temperature phase stability level is greater than or equal to 0.295° C. (“YES”), the process moves to step S547. However, in the case where that stability level is less than 0.295° C. (“NO”), the process moves to step S543.

In step S543, the evaluation unit determines whether or not the high temperature phase stability level is greater than or equal to 0.288° C. In the case where the high temperature phase stability level is greater than or equal to 0.288° C. (“YES”), the process moves to step S544. However, in the case where that stability level is less than 0.288° C. (“NO”), the process moves to step S553.

In step S544, the evaluation unit determines whether or not the number of days in the high temperature phase in the menstrual cycle is greater than or equal to nine days. When the number of days in the high temperature phase is greater than or equal to nine days (“YES”), the process moves to step S545. However, when the number of days in the high temperature phase is less than nine days (“NO”), the process moves to step S553.

In step S545, the evaluation unit determines whether or not the post-rise fall number of days is less than three days. In the case where the post-rise fall number of days is less than three days (“YES”), the process moves to step S546. However, in the case where the post-rise fall number of days is greater than or equal to three days (“NO”), the process moves to step S553.

In step S546, the evaluation unit determines the evaluation of luteum function to be “normal or insufficient (with ovulation)”.

Meanwhile, in step S547, the evaluation unit determines whether or not the evaluation index “high temperature phase rise end day” has been successfully specified in step S4. In the case where the evaluation index “high temperature phase rise end day” has been specified in step S4 (“YES”), the process moves to step S548. However, in the case where the evaluation index “high temperature phase rise end day” has not been specified in step S4 (“NO”), the process moves to step S543.

In step S548, the evaluation unit determines whether or not the value of the secondary evaluation index PA derived in step S524 is less than or equal to 9. In the case where the value of PA is less than or equal to 9 (“YES”), the process moves to step S549. However, in the case where that value is greater than 9 (“NO”), the process moves to step S543.

In step S549, the evaluation unit determines whether or not the value of the secondary evaluation index PC derived in step S524 is less than or equal to 2. In the case where the value of PC is less than or equal to 2 (“YES”), the process moves to step S550. However, in the case where that value is greater than 2 (“NO”), the process moves to step S543.

In step S550, the evaluation unit determines whether or not the number of days in the high temperature phase in the menstrual cycle is greater than or equal to nine days. When the number of days in the high temperature phase is greater than or equal to nine days (“YES”), the process moves to step S552. However, when the number of days in the high temperature phase is less than nine days (“NO”), the process moves to step S551.

In step S551, the evaluation unit determines whether or not the high-temperature sufficient consecutive number of days is greater than or equal to eight days. In the case where that number of days is greater than or equal to eight days (“YES”), the process moves to step S552. However, in the case where that number of days is less than eight days (“NO”), the process moves to step S556.

In step S552, the evaluation unit determines the evaluation of luteum function to be “insufficient (with ovulation)”.

Referring to FIG. 9G, in step S553, the evaluation unit determines whether or not the high-temperature sufficient consecutive number of days is greater than or equal to eight days. In the case where that number of days is greater than or equal to eight days (“YES”), the process moves to step S555. However, in the case where that number of days is less than eight days (“NO”), the process moves to step S554.

In step S554, the evaluation unit determines the evaluation of luteum function to be “insufficient (with ovulation) or insufficient (no ovulation)”.

Meanwhile, in step S555, the evaluation unit determines whether or not the post-rise fall day standard value is less than 0.205° C. In the case where the post-rise fall day standard value is less than 0.205° C. (“YES”), the process moves to step S556. However, in the case where that standard value is greater than or equal to 0.205° C. (“NO”), the process moves to step S554.

In step S556, the evaluation unit determines the evaluation of luteum function to be “insufficient (with ovulation).”

Through the processing performed in step S5 described above, the evaluation unit determines the evaluation of luteum function to be “normal”, “insufficient (with ovulation)”, “insufficient (no ovulation)”, or a sum of several of those evaluations.

In the processing performed in step S6, which will be described hereinafter, the evaluation unit determines the evaluation of items aside from luteum function using several of the plurality of evaluation indices derived in step S4.

Operations for evaluating items aside from luteum function will be described in detail next with reference to FIGS. 10A and 10B.

Referring to FIG. 10A, in step S601, the evaluation unit determines which process to move to next based on the number of days in the menstrual cycle. The process moves to step S602 when the menstrual cycle is less than or equal to 24 days (“less than or equal to 24 days”), the process moves to step S607 when the menstrual cycle is greater than or equal to 91 days (“greater than or equal to 91 days”), the process moves to step S608 when the menstrual cycle is greater than or equal to 39 days and less than or equal to 90 days (“greater than or equal to 39 days and less than or equal to 90 days”), and the process moves to step S613 when the menstrual cycle is greater than or equal to 25 days and less than or equal to 38 days (“greater than or equal to 25 days and less than or equal to 38 days”).

When the menstrual cycle is less than or equal to 24 days, in step S602, the evaluation unit evaluates the menstrual period as “epimenorrhagia”. The process then moves to step S603.

In step S603, the evaluation unit determines whether or not the number of days in the low temperature phase in the menstrual cycle is less than or equal to 11 days. When the number of days in the low temperature phase is less than or equal to 11 days (“YES”), the process moves to step S604. However, when the number of days in the low temperature phase is greater than 11 days (“NO”), the process moves to step S616.

In step S604, the evaluation unit determines whether or not the number of days in the high temperature phase in the menstrual cycle is greater than or equal to ten days. When the number of days in the high temperature phase is greater than or equal to ten days (“YES”), the process moves to step S605. However, when the number of days in the high temperature phase is less than ten days (“NO”), the process moves to step S616.

In step S605, the evaluation unit determines whether or not the evaluation of luteum function determined in step S5 includes an evaluation indicating that there is ovulation. In the case where the luteum function evaluation includes “with ovulation” (“YES”), the process moves to step S606. However, in the case where the luteum function evaluation does not include “with ovulation” (“NO”), the process moves to step S616.

In step S606, the evaluation unit evaluates the follicular phase as “short”. The process then moves to step S616.

Meanwhile, when the menstrual cycle is greater than or equal to 91 days, in step S607, the evaluation unit evaluates the menstrual period as “secondary amenorrhea”. The process then moves to step S616.

Meanwhile, when the menstrual cycle is greater than or equal to 39 days and less than or equal to 90 days, in step S608, the evaluation unit evaluates the menstrual period as “oligomenorrhea”. The process then moves to step S609.

In step S609, the evaluation unit determines whether or not the number of days in the low temperature phase in the menstrual cycle is greater than or equal to 25 days. When the number of days in the low temperature phase is greater than or equal to 25 days (“YES”), the process moves to step S610. However, when the number of days in the low temperature phase is less than 25 days (“NO”), the process moves to step S616.

In step S610, the evaluation unit determines whether or not the number of days in the high temperature phase in the menstrual cycle is less than or equal to 16 days. When the number of days in the high temperature phase is less than or equal to 16 days (“YES”), the process moves to step S611. However, when the number of days in the high temperature phase is greater than 16 days (“NO”), the process moves to step S616.

In step S611, the evaluation unit determines whether or not the evaluation of luteum function determined in step S5 includes an evaluation indicating that there is ovulation. In the case where the luteum function evaluation includes “with ovulation” (“YES”), the process moves to step S612. However, in the case where the luteum function evaluation does not include “with ovulation” (“NO”), the process moves to step S616.

In step S612, the evaluation unit evaluates the follicular phase as “long”. The process then moves to step S616.

Meanwhile, when the menstrual cycle is greater than or equal to 25 days and less than or equal to 38 days, in step S613, the evaluation unit determines whether or not the previous menstrual cycle (a menstrual cycle whose final day is the day before the starting day of the current menstrual period) is less than or equal to 90 days. When the previous menstrual cycle is less than or equal to 90 days (“YES”), the process moves to step S614. However, when the previous menstrual cycle is greater than 90 days (“NO”), the process moves to step S616.

In step S614, the evaluation unit compares the number of days in the previous menstrual cycle with the number of days in the current menstrual cycle and determines whether or not the difference between the two is greater than or equal to eight days. When the difference between the two is greater than or equal to eight days (“YES”), the process moves to step S615. However, when the difference between the two is less than eight days (“NO”), the process moves to step S616.

In step S615, the evaluation unit evaluates the menstrual period as “abnormal menstrual cycle”. The process then moves to step S616.

Referring to FIG. 10B, in step S616, the evaluation unit determines whether or not the phase number of the basal body temperature curve is monophasic. When the basal body temperature curve is biphasic (“NO”), the process moves to step S617. However, when the basal body temperature curve is monophasic (“YES”), the process moves to step S619.

In step S617, the evaluation unit determines whether or not the low temperature phase standard value is less than or equal to 35.50° C. In the case where the low temperature phase standard value is less than or equal to 35.50° C. (“YES”), the process moves to step S618. However, in the case where the low temperature phase standard value is greater than 35.50° C. (“NO”), the processing performed in step S6 ends (returns) and the process moves to step S7.

In step S618, the evaluation unit evaluates the measurement subject's body temperature as “low”. The processing performed in step S6 then ends (returns) and the process moves to step S7. Note that in the case where this step is not passed through in the processing performed in step S6, the evaluation unit preferably evaluates the measurement subject's body temperature as “not low” (as a default value).

Meanwhile, in step S619, the evaluation unit determines whether or not an average temperature in the menstrual cycle is less than or equal to 35.50° C. In the case where the average temperature in the menstrual cycle is less than or equal to 35.50° C. (“YES”), the process moves to step S618. However, in the case where the average temperature in the menstrual cycle is greater than 35.50° C. (“NO”), the processing performed in step S6 ends (returns) and the process moves to step S7.

Through the processing performed in step S6 described thus far, the evaluation unit determines evaluations for items aside from luteum function. Although the evaluations of items aside from luteum function include evaluating the follicular phase length, the menstrual period, the measurement subject's body temperature, and so on, the evaluations are not limited to these items.

As described above, lastly, in step S7, the server 300 outputs the evaluation results for luteum function and the items aside from luteum function in a predetermined format, by causing the control unit 310 to operate as the output unit. The information outputted at this time preferably includes the values of several of the evaluation indices in addition to the evaluation results.

Thereafter, the smartphone 200 receives the data indicating the evaluation results and the values of several of the evaluation indices and, based on that data, the basal body temperature curve schematic diagram is displayed in a rendered format. However, the rendering operations described hereinafter may be carried out by the server 300, with the resulting image data being sent to the smartphone 200 and the smartphone 200 displaying the basal body temperature curve schematic diagram based on the image data received from the server 300.

Next, the rendering of the basal body temperature curve schematic diagram carried out by the control unit 210 of the smartphone 200 operating as the rendering unit will be described in detail.

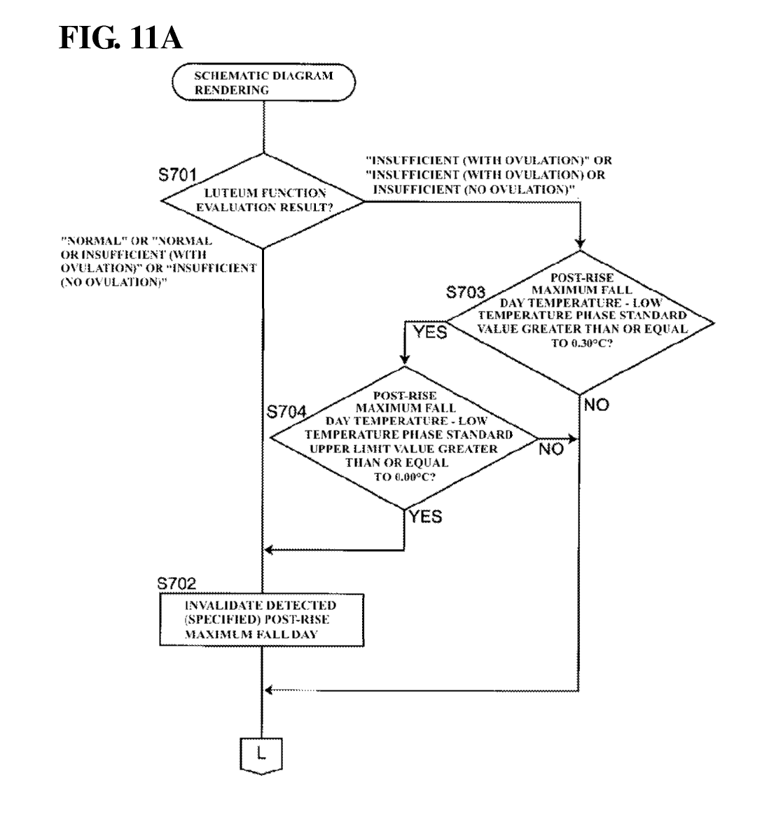

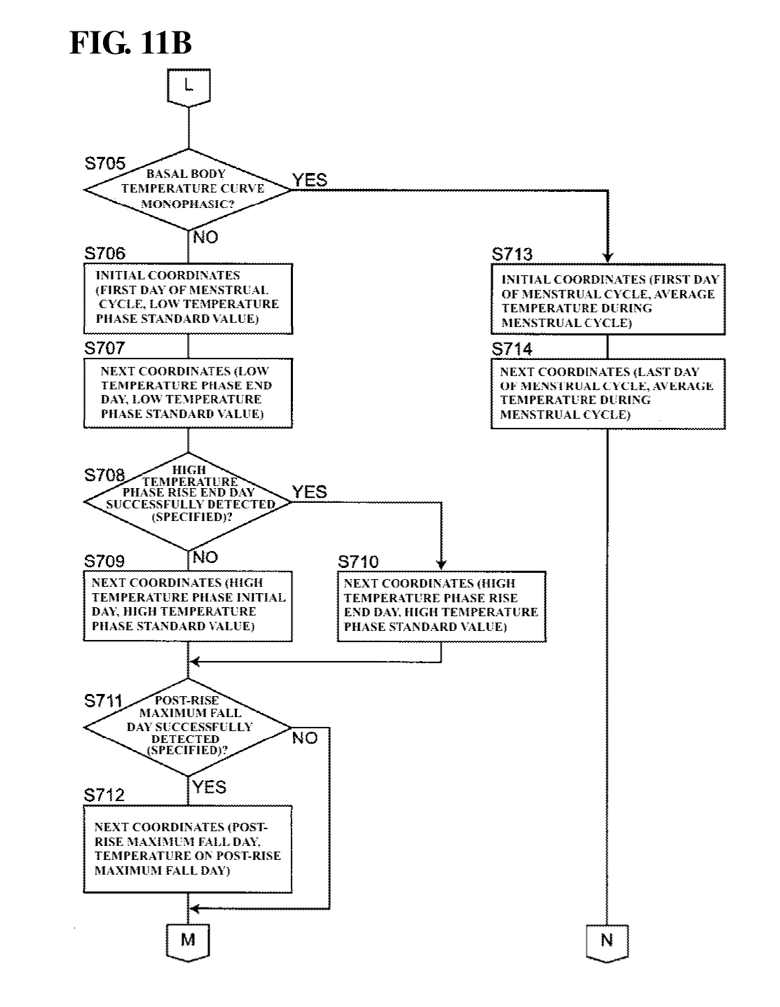

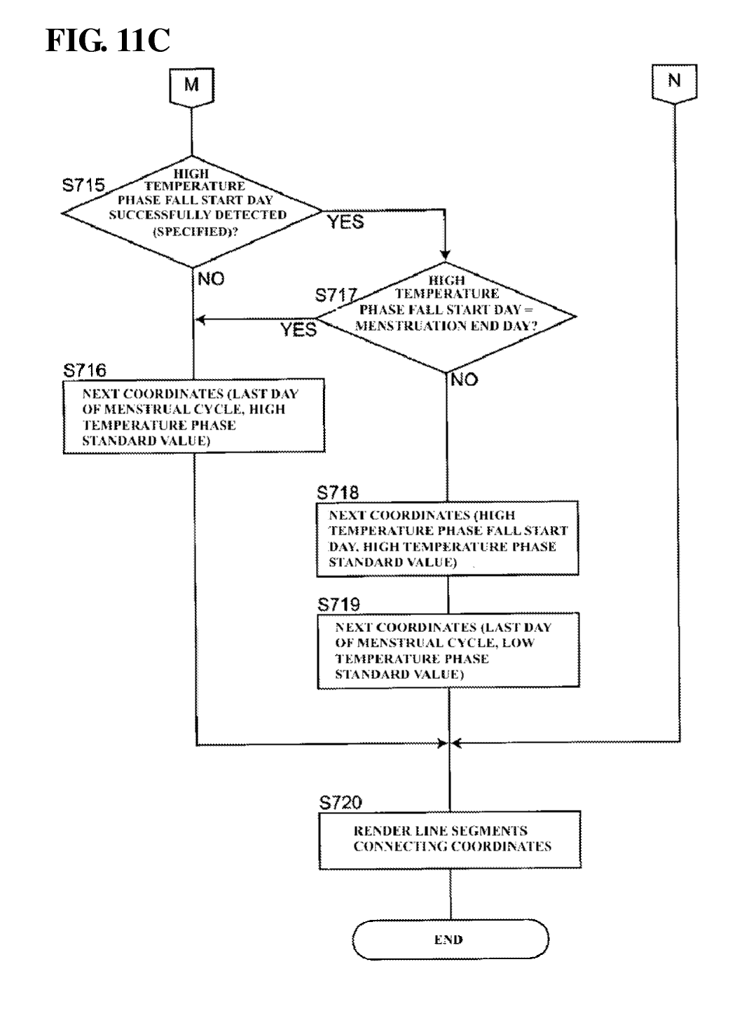

FIGS. 11A to 11C are flowcharts illustrating operations through which the control unit 210 of the smartphone 200, operating as the rendering unit, generates the image data of the basal body temperature curve schematic diagram based on the data indicating the evaluation results and several of the evaluation indices received from the server 300.

Referring to FIG. 11A, in step S701, the control unit 210 operating as the rendering unit (referred to simply as the “rendering unit” hereinafter) determines which process to move to next based on the luteum function evaluation result obtained through step S5. When the luteum function evaluation result is “insufficient (with ovulation)” or “insufficient (with ovulation) or insufficient (no ovulation)”, the process moves to step S703. On the other hand, when the luteum function evaluation result is “normal”, “normal or insufficient (with ovulation)”, or “insufficient (no ovulation)”, the process moves to step S702.

In step S702, the rendering unit invalidates the value of the evaluation index “post-rise maximum fall day” obtained from the server 300. The process then moves to step S705.

Meanwhile, in step S703, the rendering unit determines whether or not a value obtained by subtracting the value of the evaluation index “low temperature phase standard value”, obtained from the server 300, from the basal body temperature measurement value on the day indicated by the evaluation index “post-rise maximum fall day”, obtained from the server 300, is greater than or equal to 0.30° C. In the case where the value resulting from the subtraction is greater than or equal to 0.30° C. (“YES”), the process moves to step S704. However, in the case where the value resulting from the subtraction is less than 0.30° C. (“NO”), the process moves to step S705.

In step S704, the rendering unit determines whether or not a value obtained by subtracting the value of the evaluation index “low temperature phase standard upper limit value”, obtained from the server 300, from the basal body temperature measurement value on the day indicated by the evaluation index “post-rise maximum fall day”, obtained from the server 300, is greater than or equal to 0.00° C. In the case where the value resulting from the subtraction is greater than or equal to 0.00° C. (“YES”), the process moves to step S702. However, in the case where the value resulting from the subtraction is less than 0.00° C. (“NO”), the process moves to step S705.

Referring to FIG. 11B, in step S705, the rendering unit determines whether or not the evaluation index “phase number (of the basal body temperature curve)” obtained from the server 300 is monophasic. When the phase number is biphasic (“NO”), the process moves to step S706. However, when the phase number is monophasic (“YES”), the process moves to step S713.

In step S706, the rendering unit determines an initial coordinate value (a first coordinate value) to be (first day of menstrual cycle, low temperature phase standard value).

In step S707, the rendering unit determines the next coordinate value (a second coordinate value) to be (low temperature phase end day, low temperature phase standard value).

In step S708, the rendering unit determines whether or not the evaluation index “high temperature phase rise end day” has been successfully specified in step S4, based on the data obtained from the server 300. In the case where the evaluation index “high temperature phase rise end day” has been specified in step S4 (“YES”), the process moves to step S710. However, in the case where the evaluation index “high temperature phase rise end day” has not been specified in step S4 (“NO”), the process moves to step S709.

In step S709, the rendering unit determines the next coordinate value (a third coordinate value) to be (high temperature phase initial day, high temperature phase standard value).

In step S710, the rendering unit determines the next coordinate value (the third coordinate value) to be (high temperature phase rise end day, high temperature phase standard value).

In step S711, the rendering unit determines whether or not the evaluation index “post-rise maximum fall day” has been successfully specified in step S4, based on the data obtained from the server 300. In the case where the evaluation index “post-rise maximum fall day” has been specified in step S4 (“YES”), the process moves to step S712. However, in the case where the evaluation index “post-rise maximum fall day” has not been specified in step S4 (“NO”), the process moves to step S715.

In step S712, the rendering unit determines the next coordinate value (a fourth coordinate value) to be (post-rise maximum fall day, basal body temperature measurement value on post-rise maximum fall day).

Meanwhile, in step S713, the rendering unit determines the initial coordinate value (the first coordinate value) to be (first day of menstrual cycle, average temperature during menstrual cycle).

In step S714, the rendering unit determines the next coordinate value (the second coordinate value) to be (last day of menstrual cycle, average temperature during menstrual cycle).

Referring to FIG. 11C, in step S715, the rendering unit determines whether or not the evaluation index “high temperature phase fall start day” has been successfully specified in step S4, based on the data obtained from the server 300. In the case where the evaluation index “high temperature phase fall start day” has been specified in step S4 (“YES”), the process moves to step S717. However, in the case where the evaluation index “high temperature phase fall start day” has not been specified in step S4 (“NO”), the process moves to step S716.

In step S716, the rendering unit determines the next coordinate value (the fourth (or a fifth) coordinate value) to be (last day of menstrual cycle, high temperature phase standard value).

Meanwhile, in step S717, the rendering unit determines whether or not the evaluation index “high temperature phase fall start day” specified in step S4 matches the menstruation end day, based on the data obtained from the server 300. In the case where the days match (“YES”), the process moves to step S716. However, in the case where the days do not match (“NO”), the process moves to step S718.

In step S718, the rendering unit determines the next coordinate value (the fourth (or the fifth) coordinate value) to be (high temperature phase fall start day, high temperature phase standard value).

In step S719, the rendering unit determines the next coordinate value (the fifth (or a sixth) coordinate value) to be (last day of menstrual cycle, low temperature phase standard value).

In step S720, the rendering unit renders line segments connecting the plurality of coordinate values determined from step S701 to step S719, in order from the first coordinate value, to the second coordinate value, and so on.

By superimposing and displaying the basal body temperature curve schematic diagram (polygonal line graph) generated in this manner on the basal body temperature curve based on the basal body temperature measurement values, the user can intuitively understand her own luteum function and the like; as a result, the user can be expected to proactively carry out appropriate actions, such as being examined by a doctor.

In this manner, the system 100 is capable of evaluating luteum function and the like and notifying the user of the evaluation results. The user can easily, and without involving a third party, obtain knowledge regarding her own luteum function. The system 100 also presents the basal body temperature curve schematic diagram to the user. Accordingly, the user can easily and intuitively understand her own physical condition, providing an effect in which the user is encouraged to take appropriate actions (being examined by a doctor or the like) as a result.

Note that in the present specification, luteum function being “insufficient” is used to refer to all cases where luteum function is not normal, for example. In other words, a state where luteum function is insufficient includes both cases where luteum function is not normal but ovulation is occurring and cases where luteum function is not normal and no ovulation is occurring, for example.

The system 100 can be realized as a thermometer, a smartphone (a terminal device), and a computer (server), serving as hardware, and software (computer programs executed by the processors of the hardware elements).

This program can be recorded onto a recording medium such as a CD, a DVD, a flash memory, or the like as application software. By installing the application software recorded on the recording medium in what is substantially a computer device, such as a smartphone, a personal computer, a PDA (personal digital assistant), the computer device can be caused to execute the sending and receiving of the basal body temperature data, the evaluation of luteum function, the sending and receiving of luteum function evaluations, the display of luteum function evaluation results, and so on.

Note that the functionality of the server 300 may be incorporated into the smartphone 200. Furthermore, the system 100 can also be configured substantially of only the smartphone by incorporating the sensor unit 430 of the thermometer 400 into this smartphone as well.

The smartphone mentioned here may be configured of a personal computer, a tablet computer, or the like.

That is, a luteum function evaluation apparatus according to an aspect of various preferred embodiments of the present invention preferably includes a body temperature obtainment unit that obtains basal body temperature data of a measurement subject including information of basal body temperature measurement values from a plurality of days; a menstrual cycle obtainment unit that obtains information of a menstruation start day included in the plurality of days and a menstruation end day corresponding to the menstruation start day; an evaluation unit that evaluates luteum function based on a plurality of basal body temperature measurement values measured on different days within a period spanning from the menstruation start day to the menstruation end day; and an output unit that outputs a luteum function evaluation result from the evaluation unit, and the apparatus preferably further includes: a communication unit that receives, as the basal body temperature measurement value, a body temperature detected by an external thermometer from the external thermometer, and outputs the temperature to the body temperature obtainment unit; and a notifying unit that receives the luteum function evaluation result outputted by the output unit and notifies a user of the result.

Note also that the functionality of the server 300 and the smartphone 200 may be incorporated into the thermometer 400. Doing so makes it possible to substantially configure the system 100 as the thermometer alone.

That is, a luteum function evaluation apparatus according to an aspect of various preferred embodiments of the present invention preferably includes a body temperature obtainment unit that obtains basal body temperature data of a measurement subject including information of basal body temperature measurement values from a plurality of days; a menstrual cycle obtainment unit that obtains information of a menstruation start day included in the plurality of days and a menstruation end day corresponding to the menstruation start day; an evaluation unit that evaluates luteum function based on a plurality of basal body temperature measurement values measured on different days within a period spanning from the menstruation start day to the menstruation end day; and an output unit that outputs a luteum function evaluation result from the evaluation unit, and the apparatus preferably further includes: a sensor unit that detects a body temperature; and a notifying unit that receives the luteum function evaluation result outputted by the output unit and notifies a user of the result, the body temperature obtainment unit obtaining the body temperature detected by the sensor unit as the basal body temperature measurement value.

The steps indicated in the operational flows are merely examples, and it is of course possible for the system 100 to be realized through other flows as well. Furthermore, the order and configurations of the steps can be replaced, combined, and divided as desired without departing from the essential spirit of the present invention.

With respect to the evaluation of the length of the follicular phase carried out in steps S606, S612, and so on of FIG. 10A, it should be noted that a range considered to be normal for the number of days in the high temperature phase and the number of days in the menstrual cycle is assumed as a normal range for the number of days in the follicular phase, and a normal range for the number of days in the follicular phase is assumed generally to be approximately 11 to 24 days. As such, the evaluation of the “length” of the follicular phase is carried out through a comparison with that normal range in the various preferred embodiments of the present invention.

Furthermore, the various preferred embodiments of the present invention assumes that the low temperature phase=the follicular phase and the high temperature phase=luteal phase. Because the number of days in the menstrual cycle=the number of days in the low temperature phase+the number of days in the high temperature phase, the number of days in the follicular phase is in the various preferred embodiments of the present invention determined preferably as the number of days in the follicular phase=the number of days in the low temperature phase=the number of days in the menstrual cycle−the number of days in the high temperature phase. In addition, the various preferred embodiments of the present invention assume 25 to 38 days as a normal range for the number of days in the menstrual cycle, for example. Furthermore, the various preferred embodiments of the present invention assume 11 to 16 days (with 14 days being the standard) as a normal range for the number of days in the high temperature phase, for example. Accordingly, the various preferred embodiments of the present invention assume 11 (=25−14) days to 24 (=38−14) days as a guidepost for the number of days in the follicular phase, for example.

However, all of the numerical values discussed or mentioned above in the descriptions of various preferred embodiments of the present invention are merely examples, and the present invention can be carried out with different numerical values.

For example, approximately 12 to 18 days may be taken as the guidepost for the normal range of the number of days in the follicular phase. According to one source (Japan Society of Obstetrics and Gynecology, Training Corner (Vol. 59, No. 4, 2007); http://www.jsog.or.jp/activity/pdf/kenshu_{—}59-4.pdf), “the low temperature phase is normally 12 to 18 days; a low temperature phase continuing for 20 days or more may indicate delayed follicular development due to insufficient FSH secretion”.

The numerical values referred to in the descriptions of various preferred embodiments of the present invention above are merely examples, and the present invention is not intended to be limited to the above numerical value examples.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1-18. (canceled)

19. A luteum function evaluation apparatus comprising:
a body temperature obtainment unit configured to obtain basal body temperature data of a measurement subject including information of a plurality of basal body temperature measurement values from a plurality of days;
a menstrual cycle obtainment unit configured to obtain information of a menstruation start day included in the plurality of days and a menstruation end day;
an evaluation unit configured to evaluate luteum function based on the plurality of basal body temperature measurement values measured on different days within a period spanning from the menstruation start day to the menstruation end day; and
an output unit configured to output a luteum function evaluation result from the evaluation unit; wherein
based on the plurality of basal body temperature measurement values, the evaluation unit is configured to:
derive a high temperature phase rise end day and a high temperature phase fall start day;
derive a high-temperature sufficient consecutive number of days that is a number of consecutive days in a low temperature phase in which the basal body temperature measurement value is higher than a predetermined temperature, a low temperature phase instability level that is an index indicating a level of fluctuation in the basal body temperature measurement value during the low temperature phase, and a high temperature phase stability level that is an index indicating a stability level of the basal body temperature measurement value during a high temperature phase;
derive a low temperature phase standard upper limit value that is a highest temperature of the plurality of basal body temperature measurement values in a period spanning from a day after a predetermined first number of days following the menstruation start day to a final day of the low temperature phase, a low temperature phase standard lower limit value that is a lowest temperature of the plurality of basal body temperature measurement values in the period spanning from the day after the predetermined first number of days following the menstruation start day to the final day of the low temperature phase, a low temperature phase standard value that is an average value of the low temperature phase standard upper limit value and the low temperature phase standard lower limit value, a high temperature phase standard value that is an average value of the basal body temperature measurement values in the high temperature phase, and a post-rise maximum fall day temperature that is the basal body temperature measurement value on a day in the high temperature phase in which the basal body temperature measurement value has a highest decrease from the basal body temperature measurement value on the previous day; and
evaluate the luteum function based on a number of days in a current menstrual cycle, a phasic nature of a basal body temperature curve, a number of days in the high temperature phase, the high temperature phase rise end day, the high temperature phase fall start day, the high-temperature sufficient consecutive number of days, the low temperature phase instability level, the high temperature phase stability level, the low temperature phase standard upper limit value, the low temperature phase standard lower limit value, the low temperature phase standard value, the high temperature phase standard value, and the post-rise maximum fall day temperature.

20. The luteum function evaluation apparatus according to claim 19, wherein the evaluation unit is configured to evaluate the luteum function according to a plurality of evaluation levels including at least “normal” and “insufficient”.

21. The luteum function evaluation apparatus according to claim 20, wherein in a case where the luteum function has been evaluated as “insufficient”, the evaluation unit is configured to further estimate whether or not ovulation is occurring based on the plurality of basal body temperature measurement values, and is configured to further divide the evaluation of insufficient into a plurality of classes including at least “with ovulation” and “no ovulation”.

22. The luteum function evaluation apparatus according to claim 21, wherein the evaluation unit is configured to derive the current menstrual cycle that starts on the menstruation start day based on the information of the menstruation start day and the menstruation end day, derive the phasic nature indicating whether or not the basal body temperature curve includes the high temperature phase and the low temperature phase and is thus biphasic based on the plurality of basal body temperature measurement values, and evaluate the luteum function based on the number of days in the current menstrual cycle and the phasic nature of the basal body temperature curve.

23. The luteum function evaluation apparatus according to claim 22, wherein the evaluation unit is configured to derive the number of days in the high temperature phase of the current menstrual cycle based on the plurality of basal body temperature measurement values, and evaluate the luteum function based on the number of days in the current menstrual cycle, the phasic nature of the basal body temperature curve, and the number of days in the high temperature phase.

24. The luteum function evaluation apparatus according to claim 19, wherein the evaluation unit is configured to derive the current menstrual cycle starting on the menstruation start day based on the information of the menstruation start day and the menstruation end day, and divide the current menstrual cycle into a plurality of classes including at least “epimenorrhagia”, “secondary amenorrhea”, and “oligomenorrhea” based on the number of days in the current menstrual cycle.

25. The luteum function evaluation apparatus according to claim 24, wherein
the menstrual cycle obtainment unit is configured to further obtain a number of days in a previous menstrual cycle whose final day is the day before the menstruation start day; and
the evaluation unit is configured to evaluate whether or not the menstrual cycle is an abnormal menstrual cycle by comparing the number of days in the previous menstrual cycle with the number of days in the current menstrual cycle.

26. The luteum function evaluation apparatus according to claim 24, wherein based on the plurality of basal body temperature measurement values, the evaluation unit is configured to segment the current menstrual cycle into a plurality of periods including at least a low temperature phase period and a high temperature phase period, and evaluate a length of a follicular phase based on a result of the segmenting and the current menstrual cycle.

27. The luteum function evaluation apparatus according to claim 19, further comprising:
a communication unit configured to receive, as one of the plurality of basal body temperature measurement values, a body temperature detected by an external thermometer, and output the temperature to the body temperature obtainment unit; and
a notifying unit configured to receive the luteum function evaluation result outputted by the output unit and notify a user of the luteum function evaluation result.

28. The luteum function evaluation apparatus according to claim 19, further comprising:
a sensor unit configured to detect a body temperature; and
a notifying unit configured to receive the luteum function evaluation result outputted by the output unit and notify a user of the luteum function evaluation result; wherein
the body temperature obtainment unit is configured to obtain the body temperature detected by the sensor unit as one of the plurality of basal body temperature measurement values.

29. A luteum function evaluation system comprising:
a thermometer including a sensor unit configured to detect a body temperature and a first communication unit configured to send the body temperature detected by the sensor unit to an exterior as a basal body temperature measurement value;
a terminal device including a second communication unit configured to receive the basal body temperature measurement value outputted by the first communication unit, a first network communication unit configured to send the received basal body temperature measurement value to a network and receive a luteum function evaluation result from the network, and a notifying unit configured to notify a user of the luteum function evaluation result; and
a luteum function evaluation apparatus including a body temperature obtainment unit configured to obtain basal body temperature data of a measurement subject including information of basal body temperature measurement values from a plurality of days, a menstrual cycle obtainment unit configured to obtain information of a menstruation start day included in the plurality of days and a menstruation end day, an evaluation unit configured to evaluate luteum function based on a plurality of basal body temperature measurement values measured on different days within a period spanning from the menstruation start day to the menstruation end day, an output unit configured to output the luteum function evaluation result from the evaluation unit, and a second network communication unit configured to receive the basal body temperature measurement values over the network and send the luteum function evaluation result outputted by the output unit to the network; wherein
based on the plurality of basal body temperature measurement values, the evaluation unit is configured to:
derive a high temperature phase rise end day and a high temperature phase fall start day;
derive a high-temperature sufficient consecutive number of days that is a number of consecutive days in a low temperature phase in which the basal body temperature measurement value is higher than a predetermined temperature, a low temperature phase instability level that is an index indicating a level of fluctuation in the basal body temperature measurement value during the low temperature phase, and a high temperature phase stability level that is an index indicating a stability level of the basal body temperature measurement value during a high temperature phase;
derive a low temperature phase standard upper limit value that is a highest temperature of the plurality of basal body temperature measurement values in a period spanning from a day after a predetermined first number of days following the menstruation start day to a final day of the low temperature phase, a low temperature phase standard lower limit value that is a lowest temperature of the plurality of basal body temperature measurement values in the period spanning from the day after the predetermined first number of days following the menstruation start day to the final day of the low temperature phase, a low temperature phase standard value that is an average value of the low temperature phase standard upper limit value and the low temperature phase standard lower limit value, a high temperature phase standard value that is an average value of the basal body temperature measurement values in the high temperature phase, and a post-rise maximum fall day temperature that is the basal body temperature measurement value on a day in the high temperature phase in which the basal body temperature measurement value has a highest decrease from the basal body temperature measurement value on the previous day; and
evaluate the luteum function based on a number of days in a current menstrual cycle, a phasic nature of a basal body temperature curve, a number of days in the high temperature phase, the high temperature phase rise end day, the high temperature phase fall start day, the high-temperature sufficient consecutive number of days, the low temperature phase instability level, the high temperature phase stability level, the low temperature phase standard upper limit value, the low temperature phase standard lower limit value, the low temperature phase standard value, the high temperature phase standard value, and the post-rise maximum fall day temperature.

30. The luteum function evaluation system according to claim 29, wherein
the terminal device further includes an operating unit configured to accept input from the user and send information of the menstruation start day and the menstruation end day inputted by the user from the first network communication unit to the network; and
the menstrual cycle obtainment unit of the luteum function evaluation apparatus is configured to obtain the information of the menstruation start day and the menstruation end day, sent by the terminal device, from the network via the second network communication unit.

31. The luteum function evaluation system according to claim 29, wherein the terminal device further includes a rendering unit configured to generate an image and a display unit capable of displaying an image, and the rendering unit is configured to generate an image of a basal body temperature curve schematic diagram based on the luteum function evaluation result and cause the schematic diagram to be displayed in the display unit.

32. A luteum function evaluation method carried out by a luteum function evaluation apparatus, the method comprising:
a body temperature obtainment step of obtaining basal body temperature data of a measurement subject including information indicating a plurality of basal body temperature measurement values from a plurality of days;
a menstrual cycle obtainment step of obtaining information of a menstruation start day included in the plurality of days and a menstruation end day;
an evaluation step of evaluating luteum function based on the plurality of basal body temperature measurement values measured on different days within a period spanning from the menstruation start day to the menstruation end day; and
an output step of outputting a luteum function evaluation result generated in the evaluation step; wherein
the evaluation step includes substeps of, based on the plurality of basal body temperature measurement values:
deriving a high temperature phase rise end day and a high temperature phase fall start day;
deriving a high-temperature sufficient consecutive number of days that is a number of consecutive days in a low temperature phase in which the basal body temperature measurement value is higher than a predetermined temperature, a low temperature phase instability level that is an index indicating a level of fluctuation in the basal body temperature measurement value during the low temperature phase, and a high temperature phase stability level that is an index indicating a stability level of the basal body temperature measurement value during a high temperature phase;
deriving a low temperature phase standard upper limit value that is a highest temperature of the plurality of basal body temperature measurement values in a period spanning from a day after a predetermined first number of days following the menstruation start day to a final day of the low temperature phase, a low temperature phase standard lower limit value that is a lowest temperature of the plurality of basal body temperature measurement values in the period spanning from the day after the predetermined first number of days following the menstruation start day to the final day of the low temperature phase, a low temperature phase standard value that is an average value of the low temperature phase standard upper limit value and the low temperature phase standard lower limit value, a high temperature phase standard value that is an average value of the basal body temperature measurement values in the high temperature phase, and a post-rise maximum fall day temperature that is the basal body temperature measurement value on a day in the high temperature phase in which the basal body temperature measurement value has a highest decrease from the basal body temperature measurement value on the previous day; and
evaluating the luteum function based on a number of days in a current menstrual cycle, a phasic nature of a basal body temperature curve, a number of days in the high temperature phase, the high temperature phase rise end day, the high temperature phase fall start day, the high-temperature sufficient consecutive number of days, the low temperature phase instability level, the high temperature phase stability level, the low temperature phase standard upper limit value, the low temperature phase standard lower limit value, the low temperature phase standard value, the high temperature phase standard value, and the post-rise maximum fall day temperature.

33. A non-transitory computer-readable medium including a luteum function evaluation program that, when executed by a computer of a luteum function evaluation apparatus, causes the computer to execute:
a body temperature obtainment step of obtaining basal body temperature data of a measurement subject including information indicating a plurality of basal body temperature measurement values from a plurality of days;
a menstrual cycle obtainment step of obtaining information of a menstruation start day included in the plurality of days and a menstruation end day;
an evaluation step of evaluating luteum function based on the plurality of basal body temperature measurement values measured on different days within a period spanning from the menstruation start day to the menstruation end day; and
an output step of outputting a luteum function evaluation result generated in the evaluation step; wherein
the evaluation step includes substeps of, based on the plurality of basal body temperature measurement values:
deriving a high temperature phase rise end day and a high temperature phase fall start day;
deriving a high-temperature sufficient consecutive number of days that is a number of consecutive days in a low temperature phase in which the basal body temperature measurement value is higher than a predetermined temperature, a low temperature phase instability level that is an index indicating a level of fluctuation in the basal body temperature measurement value during the low temperature phase, and a high temperature phase stability level that is an index indicating a stability level of the basal body temperature measurement value during a high temperature phase;
deriving a low temperature phase standard upper limit value that is a highest temperature of the plurality of basal body temperature measurement values in a period spanning from a day after a predetermined first number of days following the menstruation start day to a final day of the low temperature phase, a low temperature phase standard lower limit value that is a lowest temperature of the plurality of basal body temperature measurement values in the period spanning from the day after the predetermined first number of days following the menstruation start day to the final day of the low temperature phase, a low temperature phase standard value that is an average value of the low temperature phase standard upper limit value and the low temperature phase standard lower limit value, a high temperature phase standard value that is an average value of the basal body temperature measurement values in the high temperature phase, and a post-rise maximum fall day temperature that is the basal body temperature measurement value on a day in the high temperature phase in which the basal body temperature measurement value has a highest decrease from the basal body temperature measurement value on the previous day; and
evaluating the luteum function based on a number of days in a current menstrual cycle, a phasic nature of a basal body temperature curve, a number of days in the high temperature phase, the high temperature phase rise end day, the high temperature phase fall start day, the high-temperature sufficient consecutive number of days, the low temperature phase instability level, the high temperature phase stability level, the low temperature phase standard upper limit value, the low temperature phase standard lower limit value, the low temperature phase standard value, the high temperature phase standard value, and the post-rise maximum fall day temperature.
